(19) United States
(12) Patent Application Publication
(10) Pub. No.: US 2026/0174931 A1
(43) Pub. Date: Jun. 25, 2026

(54) METHODS FOR CELL EXPANSION, DIFFERENTIATION, HARVESTING, ACTIVATION, OR ANY COMBINATION THEREOF USING HOLLOW FIBER MEMBRANES

(71) Applicant: Terumo BCT, Inc., Lakewood, CO (US)

(72) Inventors: Mark E. JONES, Littleton, CO (US); Nathan D. Frank, Lakewood, CO (US); Mindy M. Miller, Lakewood, CO (US); Ann Marie W. Cunningham, Lakewood, CO (US); Dalip Sethi, Thornton, CO (US)

(21) Appl. No.: 19/540,026

(22) Filed: Feb. 13, 2026

Related U.S. Application Data
(63) Continuation-in-part of application No. 18/234,470, filed on Aug. 16, 2023.
(60) Provisional application No. 63/403,592, filed on Sep. 2, 2022.

(51) Int. Cl. A61L 27/36; A61K 38/00; B01D 69/08

(52) U.S. Cl. CPC A61L 27/3633; A61K 38/00; B01D 69/08

(57) ABSTRACT

A method for functionalizing a hollow fiber membrane for cell expansion, differentiation, harvesting, activation, or any combination thereof of target cells (e.g., natural killer cells) includes contacting a biotinylated molecule to a surface of the hollow fiber membrane including an extracellular matrix component, the biotinylated molecule binding to the extracellular matrix component and having an affinity for the target cells. The biotinylated molecule is selected from the group consisting of: cytokine, epitope, ligand, monoclonal antibody, stains, aptamer, and combinations thereof. The extracellular matrix component selected from the group consisting of: fibronectin, vitronectin, fibrinogen, collagen, laminin, streptavidin, truncations thereof, and combinations thereof.

### CROSS-REFERENCE TO RELATED APPLICATIONS

This application claims the benefit of U.S. Provisional Application No. 63/403,592 filed on Sep. 2, 2022 and U.S. application Ser. No. 18/234,470 filed on Aug. 16, 2023. The entire disclosures of the above applications are incorporated herein by reference.

### FIELD

The present disclosure relates to methods for expansion, differentiation, harvesting, activation, or any combination thereof of cells using hollow fiber membranes.

### BACKGROUND

This section provides background information related to the present disclosure which is not necessarily prior art.

Natural killer (NK) cells are innate lymphoid cells that naturally attack certain cells and because of such are interesting candidates for various cell therapies and the treatment of a variety of malignant diseases. For example, natural killer cells have recently been used as cell types for engineered Chimeric Antigen Receptor (CAR) cancer therapies. Despite their promise, widespread clinical success of natural killer cell therapies has been limited because of challenges in easily and efficiently manufacturing large doses of natural killer cells. Current methods for natural killer cell expansion are often flask based, which can be time consuming, as well as expensive, and have relatively low success rates. Accordingly, it would be desirable to develop improved methods for expansion of natural killer cells and other adherent and non-adherent cells that have improved success rates and that are also less time consuming and expensive.

### SUMMARY

This section provides a general summary of the disclosure and is not a comprehensive disclosure of its full scope or all of its features.

At least one example embodiment relates to a method for functionalizing a hollow fiber membrane for cell expansion, differentiation, harvesting, activation, or any combination thereof of target cells.

In at least one example embodiment, the method may include contacting a biotinylated molecule and a surface of the hollow fiber membrane including an extracellular matrix component, where the biotinylated molecule binds to the extracellular matrix component and has an affinity for the target cells.

In at least one example embodiment, the biotinylated molecule may be selected from the group consisting of: cytokine, epitope, ligand, monoclonal antibody, stains, aptamer, chemokine, biotin, and combinations thereof.

In at least one example embodiment, the cytokine may include interleukin-21.

In at least one example embodiment, the extracellular matrix component may be selected from the group consisting of: fibronectin, vitronectin, fibrinogen, collagen, laminin, truncations thereof, and combinations thereof.

In at least one example embodiment, the extracellular matrix component may include an extracellular including the extracellular matrix component-streptavidin coupling, where the extracellular protein of the extracellular protein-streptavidin coupling binds to the surface of the hollow fiber membrane, and the streptavidin of the extracellular protein-streptavidin coupling binds to the biotinylated molecule.

In at least one example embodiment, the hollow fiber member may include a mass ratio of the streptavidin to the biotinylated molecule of greater than or equal to about 1:3 to less than or equal to about 1:9.

In at least one example embodiment, the extracellular protein-streptavidin coupling may include a fibronectin-streptavidin coupling. The fibronectin may have a molecular weight greater than or equal to about 440 kDa to less than or equal to about 500 kDa. The streptavidin may have a molecular weight greater than or equal to about 53 kDa to less than or equal to about 55 kDa.

In at least one example embodiment, the method may further include preparing the fibronectin-streptavidin coupling.

In at least one example embodiment, the preparing of the fibronectin-streptavidin coupling may include reconstituting lyophilized fibronectin with streptavidin by immerging the lyophilized fibronectin and streptavidin in water.

In at least one example embodiment, the preparing of the fibronectin-streptavidin coupling may include covalently coupling the fibronectin and the streptavidin.

In at least one example embodiment, the method may further include contacting the extracellular matrix component and the surface of the hollow fiber membrane.

In at least one example embodiment, the extracellular matrix component may be contacted with the surface of the hollow fiber membrane for a period greater than or equal to about 4 hours to less than or equal to about 24 hours prior to the contacting of the biotinylated molecule and the surface.

In at least one example embodiment, after the period, and prior to the contacting of the biotinylated molecule and the surface, the method may further include washing the hollow fiber membrane to remove any unreacted and excess portions of the extracellular matrix component.

In at least one example embodiment, the target cells may include natural killer cells.

In at least one example embodiment, the surface may be an interior-facing surface of the hollow fiber membrane.

In at least one example embodiment, the surface may be an exterior-facing surface of the hollow fiber membrane or a combination of an interior-facing surface of the hollow fiber membrane and the exterior-facing surface.

At least one example embodiment relates to another method for functionalizing a hollow fiber membrane for cell expansion, differentiation, harvesting, activation, or any combination thereof of target cells.

In at least one example embodiment, the method may include contacting an extracellular protein-streptavidin coupling and a hollow fiber membrane and contacting a biotinylated molecule and the hollow fiber membrane. The extracellular protein of the extracellular protein-streptavidin coupling may bind to the hollow fiber membrane and the streptavidin of the extracellular protein-streptavidin coupling may bind to the extracellular matrix component. The biotinylated molecule may bind to the streptavidin of the extracellular protein-streptavidin coupling. The biotinylated molecule may be selected from the group consisting of: cytokine, epitope, ligand, monoclonal antibody, stains, aptamer, chemokine, biotin, and combinations thereof.

In at least one example embodiment, the extracellular protein of the extracellular protein-streptavidin coupling may be selected from the group consisting of: fibronectin, vitronectin, fibrinogen, collagen, laminin, truncations thereof, and combinations thereof.

In at least one example embodiment, the extracellular protein-streptavidin coupling may include a fibronectin-streptavidin coupling, and the method may further include preparing the fibronectin-streptavidin coupling. The preparing of the fibronectin-streptavidin coupling may include reconstituting lyophilized fibronectin with streptavidin by immerging the lyophilized fibronectin and streptavidin in water or covalent coupling the fibronectin and the streptavidin.

In at least one example embodiment, the extracellular protein-streptavidin coupling may be contacted with the hollow fiber membrane for a period greater than or equal to about 4 hours to less than or equal to about 24 hours prior to the contacting of the biotinylated molecule and the hollow fiber membrane, and the method may further includes, prior to the contacting of the biotinylated molecule and the hollow fiber membrane, washing the hollow fiber membrane to remove any unreacted and excess portions of the extracellular protein-streptavidin coupling.

At least one example embodiment relates to another method for functionalizing a hollow fiber membrane for cell expansion, differentiation, harvesting, activation, or any combination thereof of target cells.

In at least one example embodiment, the method may include contacting a biotinylated molecule and a surface of the hollow-fiber membrane, where the surface of the hollow-fiber membrane includes fibronectin. The biotinylated molecule may bind to the fibronectin component and may have an affinity for natural killer cells. The biotinylated molecule may be selected from the group consisting of: cytokine, epitope, ligand, monoclonal antibody, stains, aptamer, biotin, and combinations thereof.

In at least one example embodiment, the fibronectin component may include a fibronectin-streptavidin coupling. The fibronectin of the fibronectin-streptavidin coupling may bind to the interior-facing surface of the hollow-fiber membrane. The streptavidin of the fibronectin-streptavidin coupling may bind to the biotinylated molecule.

In at least one example embodiment, the hollow fiber member may include a mass ratio of the streptavidin to the biotinylated molecule of greater than or equal to about 1:3 to less than or equal to about 1:9.

In at least one example embodiment, the fibronectin may have a molecular weight greater than or equal to about 440 kDa to less than or equal to about 500 kDa.

In at least one example embodiment, the streptavidin may have a molecular weight greater than or equal to about 53 kDa to less than or equal to about 55 kDa.

In at least one example embodiment, the method may further include preparing the fibronectin-streptavidin coupling.

In at least one example embodiment, the preparing of the fibronectin-streptavidin coupling may include reconstituting lyophilized fibronectin with streptavidin by immerging the lyophilized fibronectin and streptavidin in water.

In at least one example embodiment, the preparing of the fibronectin-streptavidin coupling may include inducing covalent coupling of the fibronectin and the streptavidin.

In at least one example embodiment, the cytokine may include interleukin-21.

In at least one example embodiment, the method may further include contacting the fibronectin component and the interior-facing surface of the hollow-fiber membrane.

In at least one example embodiment, the fibronectin component may be contacted with interior-facing surface of the hollow-fiber membrane for a period greater than or equal to about 4 hours to less than or equal to about 24 hours prior to the contacting of the biotinylated molecule and the interior-facing surface.

In at least one example embodiment, after the period and prior to the contacting of the biotinylated molecule and the interior-facing surface, the hollow-fiber membrane may be washed to remove an unreacted and excess portion of the fibronectin component.

In at least one example embodiment, the fibronectin component may include plasma-derived fibronectin or recombinant fibronectin.

At least one example embodiment relates to another method for functionalizing a hollow fiber membrane for cell expansion, differentiation, harvesting, activation, or any combination thereof of target cells.

In at least one example embodiment, the method may include contacting a biotinylated molecule and the hollow fiber membrane including an extracellular matrix component. The biotinylated molecule may bind to the extracellular matrix component and may have an affinity for the target cells.

In at least one example embodiment, the biotinylated molecule may be selected from the group consisting of: cytokine, epitope, ligand, monoclonal antibody, stains, aptamer, chemokine, biotin, and combinations thereof.

In at least one example embodiment, the extracellular matrix component may be selected from the group consisting of: fibronectin, vitronectin, fibrinogen, collagen, laminin, streptavidin, truncations thereof, and combinations thereof.

In at least one example embodiment, the extracellular matrix component may include an extracellular protein-streptavidin coupling. The extracellular protein of the extracellular protein-streptavidin coupling may bind to a target surface of the hollow fiber membrane. The streptavidin of the extracellular protein-streptavidin coupling may bind to the biotinylated molecule.

In at least one example embodiment, the hollow fiber member may include a mass ratio of the streptavidin to the biotinylated molecule of greater than or equal to about 1:3 to less than or equal to about 1:9.

In at least one example embodiment, the extracellular protein-streptavidin coupling may include a fibronectin-streptavidin coupling. The fibronectin may have a molecular weight greater than or equal to about 440 kDa to less than or equal to about 500 kDa. The streptavidin may have a molecular weight greater than or equal to about 53 kDa to less than or equal to about 55 kDa.

In at least one example embodiment, the method may further include preparing the fibronectin-streptavidin coupling.

In at least one example embodiment, the preparing of the fibronectin-streptavidin coupling may include reconstituting lyophilized fibronectin with streptavidin by immerging the lyophilized fibronectin and streptavidin in water.

In at least one example embodiment, the preparing of the fibronectin-streptavidin coupling may include covalently coupling the fibronectin and the streptavidin.

In at least one example embodiment, the method may further include preparing the extracellular protein-streptavidin coupling. The preparing of the extracellular protein-streptavidin coupling may include contacting the extracellular protein and a precursor hollow fiber membrane and, after the contacting of the extracellular protein and the precursor hollow fiber membrane, contacting the streptavidin and the precursor hollow-fiber membrane including the extracellular protein to form the extracellular protein-streptavidin coupling and the hollow fiber membrane including the extracellular matrix component. The extracellular protein-streptavidin coupling may include both classical and non-classical hydrogen bonding between the extracellular protein and the streptavidin.

In at least one example embodiment, the extracellular protein may be contacted with the precursor hollow fiber membrane for a first period greater than or equal to about 4 hours to less than or equal to about 24 hours.

In at least one example embodiment, the method may further include, after the first period and before the contacting of the streptavidin and the precursor hollow-fiber membrane, washing the precursor hollow fiber membrane to remove any unreacted and excess portions of the extracellular protein.

In at least one example embodiment, the streptavidin may be contacted to the precursor hollow fiber membrane for a second period greater than or equal to about 4 hours to less than or equal to about 24 hours.

In at least one example embodiment, the method may further include, after the second period and before the contacting of the biotinylated molecule and the hollow fiber membrane including the extracellular matrix component, washing the hollow fiber membrane including the extracellular matrix component to remove any unreacted and excess portions of the streptavidin.

In at least one example embodiment, the method may further include contacting the extracellular matrix component and a precursor hollow fiber membrane to form the hollow fiber membrane including the extracellular matrix component.

In at least one example embodiment, the extracellular matrix component may be contacted with the surface of the hollow fiber membrane for a period greater than or equal to about 4 hours to less than or equal to about 24 hours prior to the contacting of the biotinylated molecule and the surface.

In at least one example embodiment, after the period and prior to the contacting of the biotinylated molecule and the surface, the method may further include washing the hollow fiber membrane to remove any unreacted and excess portions of the extracellular matrix component.

At least one example embodiment relates to another method for functionalizing a hollow fiber membrane for cell expansion, differentiation, harvesting, activation, or any combination thereof of target cells.

In at least one example embodiment, the method may include contacting an extracellular protein and a surface of a hollow fiber membrane; after the contacting of the extracellular protein and the surface of the hollow fiber membrane, contacting streptavidin and the surface of the hollow fiber membrane, the streptavidin forming extracellular protein-streptavidin coupling with the extracellular protein, the extracellular protein-streptavidin coupling including both classical and non-classical hydrogen bonding between the extracellular protein and the streptavidin; and after the contacting of the streptavidin and the surface of the hollow fiber membrane, contacting a biotinylated molecule and the surface of hollow fiber membrane, the biotinylated molecule binding to the extracellular protein of the extracellular protein-streptavidin coupling and having an affinity for the target cells.

In at least one example embodiment, the method may further include, after the contacting of the extracellular protein and the surface of the hollow fiber membrane and before the contacting of the streptavidin and the surface of the hollow fiber membrane, washing the surface of hollow fiber membrane to remove any unreacted and excess portions of the extracellular protein.

In at least one example embodiment, the method may further include, after the contacting of the streptavidin and the surface of the hollow fiber membrane and before the contacting of the biotinylated molecule and the surface of the hollow fiber membrane, washing the surface of hollow fiber membrane to remove any unreacted and excess portions of the streptavidin.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example cell expansion system having a bioreactor in accordance with at least one example embodiment;

FIG. 2 is an illustration of an example hollow fiber membrane which may be incorporated into a cell expansion system like the cell expansion system illustrated in FIG. 1, in accordance with at least one example embodiment;

FIG. 3 is an illustration of an example bioreactor that shows circulation paths through the bioreactor, and which may be incorporated into a cell expansion system like the cell expansion system illustrated in FIG. 1, in accordance with at least one example embodiment;

FIG. 4 is an illustration of an example rocking device configured to move a bioreactor in accordance with at least one example embodiment;

FIG. 5 is a perspective view of another example cell expansion system having a bioreactor and a premounted fluid conveyance device in accordance with at least one example embodiment;

FIG. 6 is a perspective view of an example housing for the example cell expansion system as illustrated in FIG. 5;

FIG. 7 is a perspective view of the premounted fluid conveyance device as illustrated in FIG. 5;

FIG. 8 is an illustration of example flow paths of an example cell expansion system, like the cell expansion system illustrated in FIG. 1 and/or the cell expansion system illustrated in FIG. 5, in accordance with at least one example embodiment of the present disclosure;

FIG. 9 is an illustration of other example flow paths of an example cell expansion system, like the cell expansion system illustrated in FIG. 1 and/or the cell expansion system illustrated in FIG. 5, in accordance with at least one example embodiment;

FIG. 10 is a flow diagram illustrating operational characteristics of an example process for expanding cells using an example cell expansion system, like the cell expansion system illustrated in FIG. 1 and/or the cell expansion system illustrated in FIG. 5, in accordance with at least one example embodiment;

FIG. 11 is a block diagram of an example processing system for use in an example cell expansion system, like the cell expansion system illustrated in FIG. 1 and/or the cell expansion system illustrated in FIG. 5, in accordance with at least one example embodiment;

FIG. 12 is a flowchart illustrating an example method for readying a bioreactor for cell expansion and/or differentiation and/or harvesting and/or activation using a biotinylated protein coupling in accordance with at least one example embodiment;

FIG. 13 is a flowchart illustrating another example method for readying a bioreactor for cell expansion and/or differentiation and/or harvesting and/or activation using a biotinylated protein coupling in accordance with at least one example embodiment; and

FIG. 14 is a flowchart illustrating an example method for readying a bioreactor for cell expansion and/or differentiation and/or harvesting and/or activation using a cytokine in accordance with at least one example embodiment.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

Example embodiments will now be described more fully with reference to the accompanying drawings.

The present disclosure relates to methods for cell expansion, differentiation, harvesting, activation, or any combination thereof using cell expansion systems like those described, for example, in U.S. Pat. No. 8,309,347, titled CELL EXPANSION SYSTEMS AND METHODS OF USE, issued on Nov. 13, 2012 and/or U.S. Pat. No. 9,677,042, titled CUSTOMIZABLE METHODS AND SYSTEMS OF GROWING AND HARVESTING CELLS IN A HOLLOW FIBER BIOREACTOR SYSTEM, issued Jun. 13, 2017 and/or U.S. Pat. No. 9,725,689, titled CONFIGURABLE METHODS AND SYSTEMS OF GROWING AND HARVESTING CELLS IN A HOLLOW FIBER BIOREACTOR SYSTEM, issued Aug. 8, 2017 and/or U.S. application Ser. No. 15/943,536, titled EXPANDING CELLS IN A BIOREACTOR, filed Apr. 2, 2018, and published Oct. 2, 2018 and/or U.S. Pat. No. 10,577,585 titled CELL EXPANSION and issued on Mar. 3, 2020, the entire disclosures of which are hereby incorporated by reference.

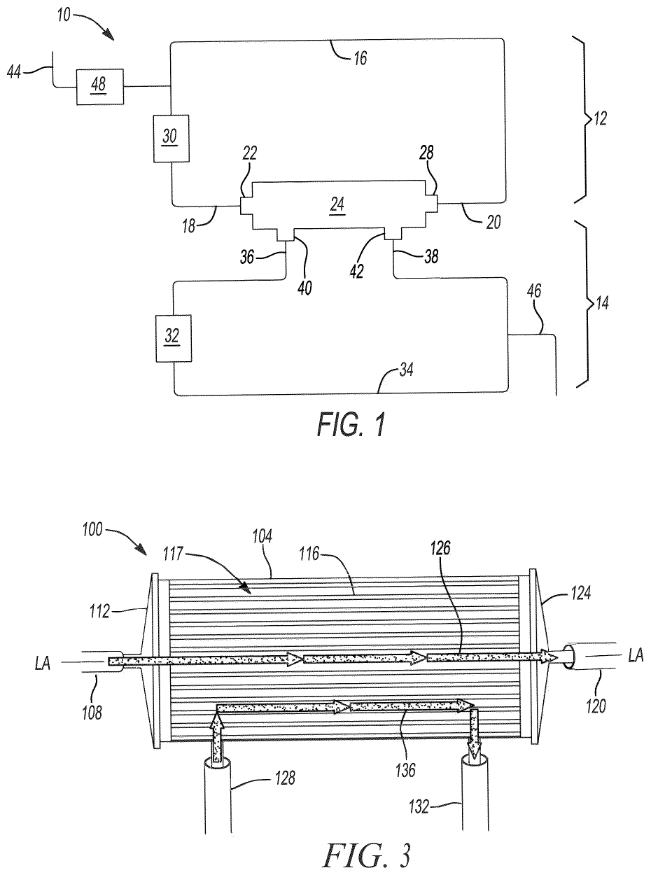

Cell expansions systems, also commonly referred to as hollow fiber bioreactors, are cell culturing systems used to expand and differentiate cells, including both adherent and non-adherent cell types. For example, FIG. 1 is an illustration of an example cell expansion system 10 having a first fluid circulation path 12 and a second fluid circulation path 14.

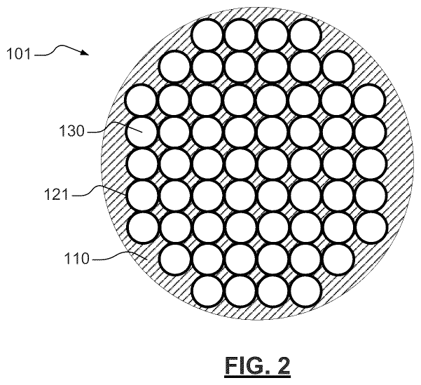

The first fluid circulation path 12 may include a first fluid flow path 16 having opposing ends 18 and 20. The first fluid flow path 16 may be in fluid communication with a cell growth chamber 24 (which can also be referred to as a “bioreactor”). For example, the first opposing end 18 of the first fluid flow path 16 may be in fluid communication with a first inlet 22 of the cell growth chamber 24, and the second opposing end 20 may be in fluid communication with first outlet 28 of the cell growth chamber 24. The cell growth chamber 24 may support a hollow fiber membrane (“HFM”) (see, e.g., FIG. 2) and fluid in the first circulation path 12 may flow through hollow fibers that define the hollow fiber membrane disposed in the cell growth chamber 24. In at least one example embodiment, a first fluid flow control device 30 may be operably coupled to the first fluid flow path 16 and may control the flow of fluid in first fluid circulation path 12.

The second fluid circulation path 14 may include a second fluid flow path 34 and a second fluid flow control device 32. Like the first fluid flow path 16, the second fluid flow path 34 may have opposing ends 36 and 38. The opposing ends 36 and 38 of second fluid flow path 34 may in fluid communication with an inlet port 40 and an outlet port 42 of the cell growth chamber 24. For example, a first end 36 of the second fluid flow path 34 may be in fluid communication with the inlet port 40 of the cell growth chamber 24, and the second end 38 of the second fluid flow path 34 may be in fluid communication with the outlet port 2. Fluid in the second circulation path 14 may be in contact with an exterior or outside of the hollow fiber membrane disposed in the cell growth chamber 24. In at least one example embodiment, a second fluid flow control device 32 may be operably coupled to the second fluid flow path 34 and may be configured to control the flow of fluid in and through the second fluid circulation path 14. Fluid in first fluid circulation path 12 (for example, as controlled by the first fluid flow control device 20) may flow in either a co-current or counter-current direction with respect to a fluid flow in second fluid circulation path 14 (for example, as controlled by the second fluid flow control device 32).

In at least one example embodiment, a fluid inlet path 44 may be fluidly associated with the first fluid circulation path 12, and a fluid outlet path 46 may be fluidly associated with the second fluid circulation path 14. The fluid inlet path 44 permits fluid into first fluid circulation path 12, while the fluid outlet path 46 permits fluid to exit the cell expansion system 10. In at least one example embodiment, as illustrated, a third fluid flow control device 48 may be operably associated with the fluid inlet path 44. Although not illustrated, it should be recognized that, in various example embodiments, a fourth fluid flow control device may alternatively, or additionally, be associated operably associated with the first outlet path 46. In at least one example embodiment, the fluid flow control devices (including the first fluid flow control device 30, the second fluid flow control device 32, the third fluid flow control device 48, and/or the fourth fluid flow control device) may include a pump, valve, clamp, or any combination thereof. For example, multiple pumps, valves, and clamps can be arranged in any combination. In at least one example embodiment, the fluid flow control device may be, or include, a peristaltic pump. Fluid circulation paths (including, for example, the first fluid circulation path 12 and/or the second fluid circulation path 14), inlet ports (including, for example, the fluid inlet port 44), and/or the outlet port (including, for example, the fluid outlet port 46), may include any known tubing material, and any kind of fluid—including, for example, buffers, protein containing fluid, and cell-containing fluid—can flow through the various circulation paths (including, for example, the first fluid circulation path 12 and/or the second fluid circulation path 14), inlet paths (including, for example, the fluid inlet port 44), and outlet paths (including, for example, the fluid outlet port 46). It should be recognized that the terms “fluid,” “media,” and “fluid media” are used interchangeably.

In at least one example embodiment, the first and second fluid circulation paths 12, 14 may be maintained in the cell growth chamber 24 by way of a hollow fiber membrane, where fluid in first fluid circulation path 12 flows through an intracapillary (“IC”) space of the hollow fiber membrane and fluid in the second circulation path 14 flows through the extracapillary (“EC”) space of the cell growth chamber 24. The first circulation path 12 may be referred to as the “intracapillary loop” or “intracapillary space” or “IC loop” or “intraporous space”. The second fluid circulation path 14 may be referred to as the “extracapillary loop” or “extracapillary space” or “EC loop” or “interporous space”. By way of example, FIG. 2 illustrates a cross-section of an example hollow fiber membrane 101 that includes a plurality of semi-permeable hollow fibers (also referred to as hollow columns and/or hollow matrixes) 121, where space or voids 130 within the hollow fibers 121 define an intracapillary space and a space exterior to or outside of the hollow fibers 121 defines an extracapillary space 110. In at least one example embodiment, the material used to form the hollow fiber membrane 101 may be any biocompatible polymeric material that is capable of being made into the hollow fibers 121. For example, synthetic polysulfone-based materials (e.g., polyethersulfones (PES)) are often used to form the hollow fibers 121.

FIG. 3 is an illustration of an example cell growth chamber 100 (which can also be referred to as a “bioreactor”) that may be used as the cell growth chamber 24 of the cell expansion system 10. The cell growth chamber 100 has a longitudinal axis (represented by the line LA-LA) and may include a cell growth chamber housing 104. The cell growth chamber housing 104 may have four openings or ports, including, for example, an intracapillary inlet port 108, an intracapillary outlet port 120, an extracapillary inlet port 128, and an extracapillary outlet port 132. A first fluid (which may also be referred to as an intracapillary fluid or media) in a first circulation path (like the first fluid circulation path 12) may enter the cell growth chamber 100 through the intracapillary inlet port 108 at a first fluid manifold end 112 of the cell growth chamber 100 and into and through the intracapillary spaces of a plurality of hollow fibers 116 and out of cell growth chamber 100 through intracapillary outlet port 120, which is located at a second fluid manifold end 124 of the cell growth chamber 100. The fluid path between the intracapillary inlet port 108 and the intracapillary outlet port 120 may define an intracapillary portion 126 of the cell growth chamber 100. A second fluid (which may also be referred to as an extracapillary media or fluid) in a second circulation path (like the second fluid circulation path 14) may enter the cell growth chamber 100 through the extracapillary inlet port 128. This second fluid may contact the extracapillary space, or outside, of the hollow fiber membrane 117 and exits the cell growth chamber 100 via the extracapillary outlet port 132. The fluid path between the extracapillary inlet port 128 and the extracapillary outlet port 132 may define an extracapillary portion 136 of the cell growth chamber 100.

As the second fluid comes into contact with the outside of the hollow fibers 116 small molecules (e.g., ions, water, oxygen, lactate, etc.) may diffuse through the hollow fibers 116 from the interior or intracapillary space of the hollow fibers 116 to the exterior or extracapillary space, or alternatively, or additionally, from the extracapillary space to the intracapillary space. Large molecular weight molecules (e.g., growth factors and/or proteins) are often too large to pass through the membrane walls of the hollow fibers 116 and remain in the intracapillary space (or alternatively, or additionally, in the extracapillary space) of the hollow fibers 116. The mediums defining the first and second fluids may be replaced as needed and may alternatively, or additionally, be circulated through an oxygenator and/or gas transfer module to exchange gasses, as needed. As discussed below, cells for expansion may be contained within the first fluid circulation path 12 and/or the second fluid circulation path 14 and may enter the hollow fiber cell growth chamber 100 on one or both of the intracapillary space or the extracapillary space.

In at least one example embodiment, cells (for example, cord blood derived CD34+ hematopoietic stem/progenitor cells, monocytes, macrophages, hepatocytes, and/or endothelial cells) may be seeded (for example, for expansion, differentiation, harvesting, activation, or any combination thereof) in the intracapillary space 130 of the hollow fibers 116, while a cell culture medium may be pumped through the extracapillary space 110 of the hollow fibers 116 to deliver nutrients to the cells via hollow fiber membrane perfusion during expansion, differentiation, harvesting, and/or activation. However, in various other example embodiments, cells (for example, cord blood derived CD34+ hematopoietic stem/progenitor cells, monocytes, macrophages, hepatocytes, and/or endothelial cells) may be seeded for example, for expansion, differentiation, harvesting, and/or activation) in the extracapillary space 110, while the cell culture medium may be pumped through the intracapillary space 130 to deliver nutrients to the cells via hollow fiber membrane perfusion during expansion. In various other example embodiments, cells (for example, cord blood derived CD34+ hematopoietic stem/progenitor cells, monocytes, macrophages, hepatocytes, and/or endothelial cells) may be seeded (for example, for expansion, differentiation, harvesting, and/or activation) in the intracapillary space 130, while the cell culture medium may be pumped through both the extracapillary space 110 and the intracapillary space 130. Movement of the cell culture medium through the intracapillary space 130 and/or the extracapillary space 110 may help to remove excess cells, for example, those not adhered to surfaces of the hollow fiber 116.

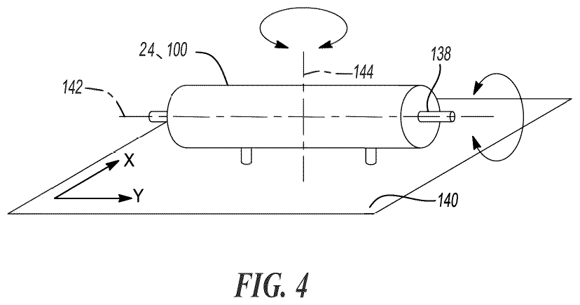

In at least one example embodiment, the cell expansion system 10 may also include a device that is configured to move or “rock” the cell growth chamber 24, 100 relative to other components of the cell expansion system 10. The device may be a rotational and/or lateral rocking device. For example, as illustrated in FIG. 4, the cell growth chamber 24, 100 may be rotationally connected to one or more rotational rocking components 138 and to a lateral rocking component 140. A first rotational rocking component 138 may be rotationally associated with the cell growth chamber 24, 100. For example, the first rotational rocking component 138 may be configured to rotate the cell growth chamber 24, 100 around a first or central rotational axis 142. In at least one example embodiment, the cell growth chamber 24, 100 may be rotated in alternating fashion, including, for example, in a first clockwise direction and then in a second counterclockwise direction around the central axis 142.

Although not illustrated, it should be recognized that, in various example embodiments, a second rotational rocking component may be configured to move the cell growth chamber 24, 100 about a second rotational axis 144 that passes through a center point of the cell growth chamber 24, 100 normal to the central axis 142. In at least one example embodiment, the cell growth chamber 24, 100 may be rotated in alternating fashion, including, for example, in a first clockwise direction and then in a second counterclockwise direction around the second axis 144. In at least one example embodiment, the cell growth chamber 24, 100 may also be rotated around the second axis 144 and positioned in a horizontal or vertical orientation relative to gravity. The lateral rocking component 140 may be laterally associated with the cell growth chamber 24, 100. For example, a plane of the lateral rocking component 140 may move laterally in the x-direction and γ-direction.

The rotational and/or lateral movement of the cell growth chamber 24, 100 may reduce the settling of cells and the likelihood of cells becoming trapped within a portion of the cell growth chamber 24, 100. In at least one example embodiment, the rate of cells settling in the cell growth chamber 24, 100 may be proportional to the density difference between the cells and the suspension media, according to Stoke's Law. In at least one example embodiment, a 180-degree rotation (fast) with a pause (having, for example, a total combined time of 30 seconds) repeated as described above may help to keep non-adherent cells (for example, t-cells and/or red blood cells) suspended. A minimum rotation of about 180-degrees may be preferred, however various degrees of rotation, including up to or greater than 360-degrees, may be used. Different rocking components may be used separately or may be combined in any combination. For example, a rocking component that rotates cell growth chamber 24, 100 around central axis 142 may be combined with the rocking component that rotates cell growth chamber 24, 100 around axis 144. Likewise, clockwise and counterclockwise rotation around different axes may be performed independently in any combination.

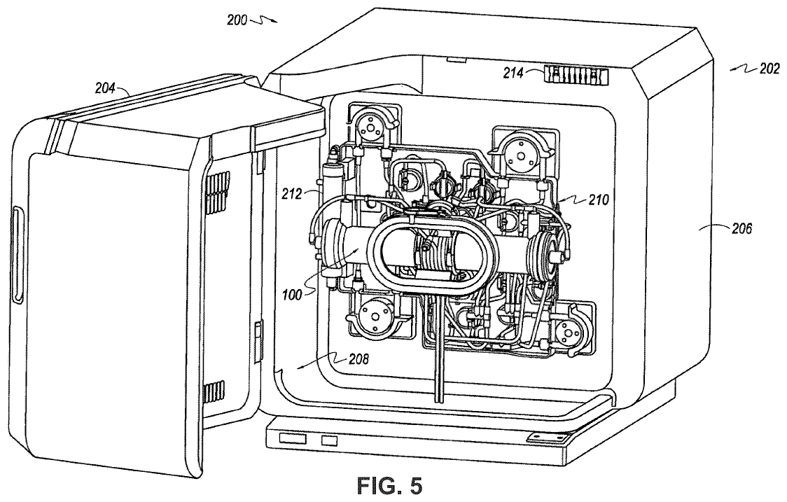

FIG. 5 is an illustration of another example cell expansion system 200. The cell expansion system 200 may be the same as the cell expansion system 10 illustrated in FIG. 1 except that the cell expansion system 200 includes a premounted fluid conveyance assembly 210. For example, the cell expansion system 200 may include a cell expansion machine 202 having a back portion 206 and a hatch or closeable door 204 that engages with the back portion 206. An interior space 208 of the cell expansion machine 202 may be configured to receive the premounted fluid conveyance assembly 210. The premounted fluid conveyance assembly 210 may be detachably attachable to the cell expansion machine 202 so as to facilitate relatively quick exchange of a new or unused premounted fluid conveyance assembly 210. For example, a cell expansion machine 202 may be operated to grow or expand a first set of cells using a first premounted fluid conveyance assembly and to be subsequently used to grow or expand a second set of cells using a second premounted fluid conveyance assembly without needing to be sanitized between the interchange of the first premounted fluid conveyance assembly 210 for the second premounted fluid conveyance assembly. In each variation, the premounted fluid conveyance assembly 210 includes a cell growth chamber (also referred to as a bioreactor and like the cell growth chamber 24 illustrated in FIG. 1 and/or the cell growth chamber 100 illustrated in FIG. 2) and an oxygenator or gas transfer module 212. The cell expansion system 200 includes a plurality of tubing guide slots 214 for receiving various media to be placed in fluid communication with the premounted fluid conveyance assembly 210.

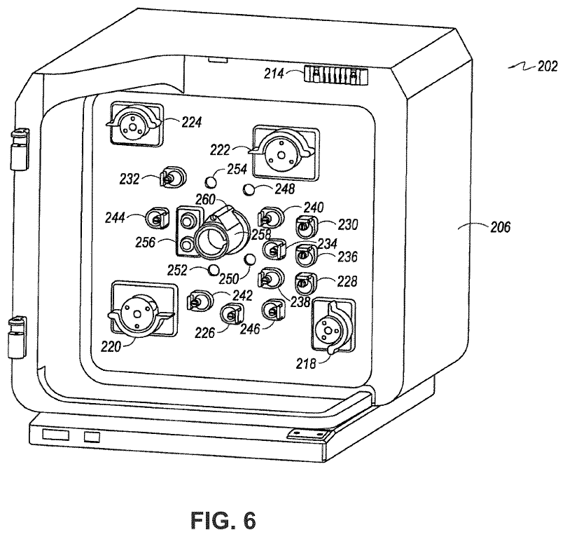

FIG. 6 is an illustration of a back portion 206 of the cell expansion machine 202 prior to detachably attaching the premounted fluid conveyance assembly 210. The closable door 204 is omitted from FIG. 6. As illustrated, the back portion 206 of the cell expansion machine 202 may include a number of different structures for working in combination with elements of the premounted fluid conveyance assembly 210. For example, the back portion 206 of the cell expansion machine 202 may include a plurality of peristaltic pumps for cooperating with pump loops of the premounted fluid conveyance assembly 210, including, for example, an intracapillary circulation pump 218, an extracapillary circulation pump 220, an intracapillary inlet pump 222, and/or an extracapillary inlet pump 224. The back portion 206 may also include a plurality of valves, including, for example, an intracapillary circulation valve 226, a reagent valve 228, an intracapillary media valve 230, an air removal valve 232, a cell inlet valve 234, a wash valve 236, a distribution valve 238, an extracapillary media valve 240, an intracapillary waste valve 242, an extracapillary waste valve 244, and/or a harvest valve 246. Several sensors may also be associated with the back portion 206 of the cell expansion machine 202, including, for example, an intracapillary outlet pressure sensor 248, a combination intracapillary inlet pressure and temperature sensors 250, a combination extracapillary inlet pressure and temperature sensors 252, and/or an extracapillary outlet pressure sensor 254. In at least one example embodiment, an optical sensor 256 for an air removal chamber may also be disposed in the back portion 206.

The back portion 206 may also include a shaft or rocker control 258 for rotating the cell growth chamber 24, 100. A shaft fitting 260 may be associated with the shaft or rocker control 258 to help ensure proper alignment of a shaft access aperture 424 of a tubing organizer 300 of an example premounted conveyance assembly (e.g., premounted conveyance assembly 400) with the back portion 206 of the cell expansion machine 202. Rotation of shaft or rocker control 258 may impart rotational movement to shaft fitting 260 and the cell growth chamber 24, 100. Thus, when an operator or user of the cell expansion system 200 attaches a new or unused premounted fluid conveyance assembly 400 to the cell expansion machine 202, the alignment is a simple matter of properly orienting the shaft access aperture 424 of the premounted fluid conveyance assembly 400 with the shaft fitting 260.

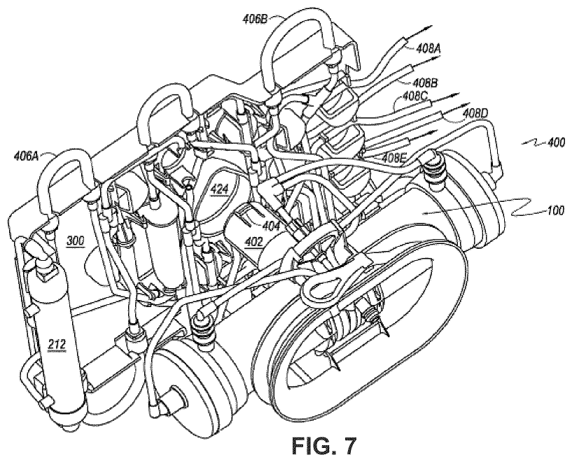

FIG. 7 is a perspective view of the example detachably-attachable premounted fluid conveyance assembly 400. The premounted fluid conveyance assembly 400 may be detachably attachable to the cell expansion machine 202 to facilitate quick placement of a new or unused premounted fluid conveyance assembly 400 in the cell expansion machine 202. The cell growth chamber 24, 100 may be attached to a bioreactor coupling that includes a shaft fitting 402. The shaft fitting 402 may include one or more shaft fastening mechanisms, such as a biased arm or spring member 404 for engaging a shaft 258 of the cell expansion machine 202.

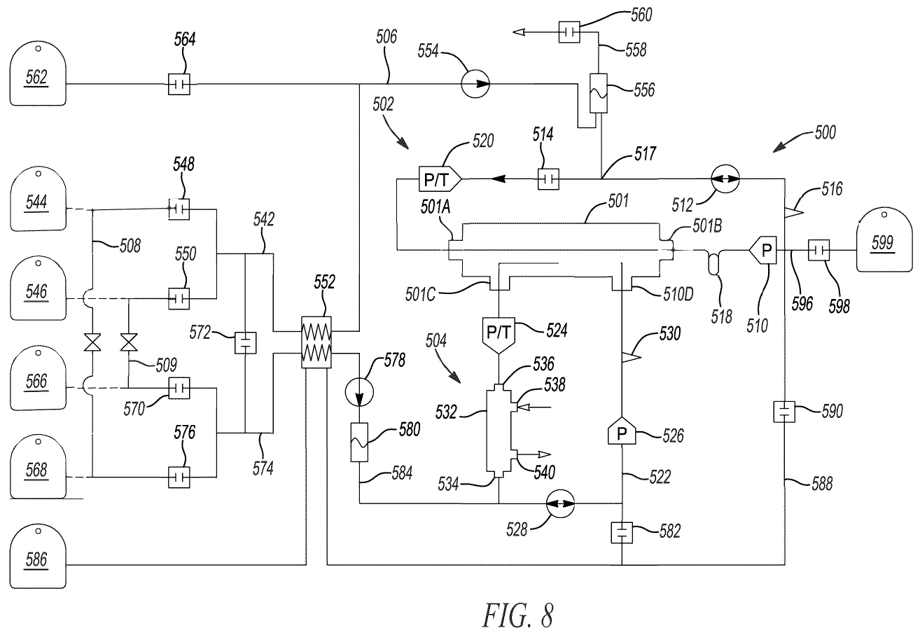

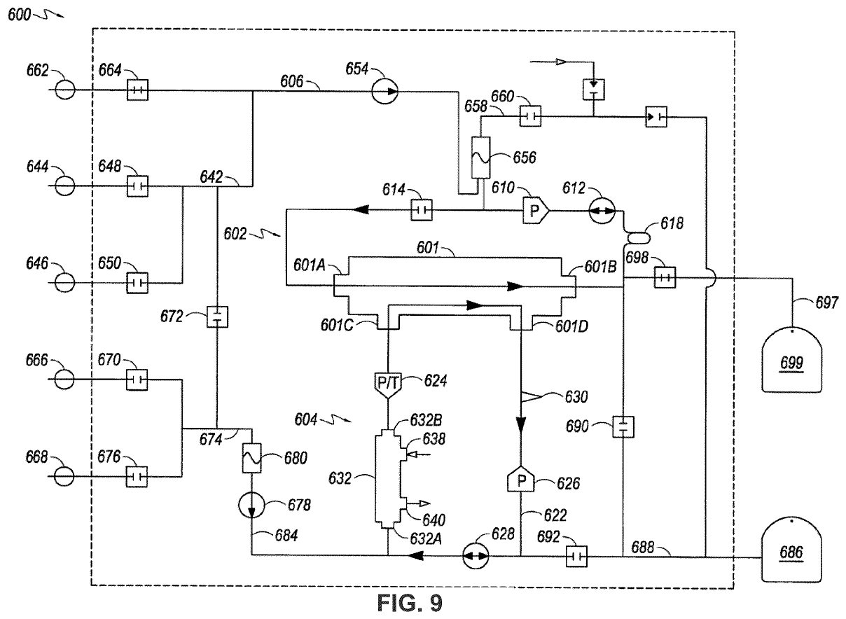

The premounted fluid conveyance assembly 400 may include a plurality of tubings (including, for example, tubings 408A, 408B, 408C, 408D, 408E) and various tubing fittings to form the fluid paths illustrated in FIGS. 8 and 9, which are discussed below. Pump loops 406A and 406B may also be provided. Although the various media can be provided at the cell expansion machine 202, in various example embodiments, the premounted fluid conveyance assembly 400 may include sufficient tubing length to extend to an exterior of the cell expansion machine 202 and to enable welded connections to tubing associated with media bag(s) or container(s).

FIG. 8 is a schematic of an example cell expansion system 500, like the cell expansion system 100 illustrated in FIG. 1 and/or the cell expansion system 200 illustrated in FIG. 5, that illustrates example flow paths. As illustrated, the cell expansion system 500 may include a first fluid circulation path 502 (also referred to as the “intracapillary loop” or “intracapillary space” or “IC loop”) and a second fluid circulation path 504 (also referred to as the “extracapillary loop” or “extracapillary space” or “EC loop”). In at least one example embodiment, the cells may be positioned in the intracapillary space 502, while a cell culture medium may be pumped through the extracapillary space 504 to deliver nutrients to the cells via hollow fiber membrane perfusion during expansion. It should be recognized, however, in at least one other example embodiment, cells can be positioned in the extracapillary space 504, while the cell culture medium may be pumped through the intracapillary space 502 to deliver nutrients to the cells via hollow fiber membrane perfusion during expansion. In at least one other example embodiment, cells may be positioned in the intracapillary space 502 while the cell culture medium may be pumped through both the extracapillary space 504 and the intracapillary space 502. In at least one other example embodiment, cells may be positioned in the extracapillary space 504 while the cell culture medium may be pumped through both the extracapillary space 504 and the intracapillary space 502.

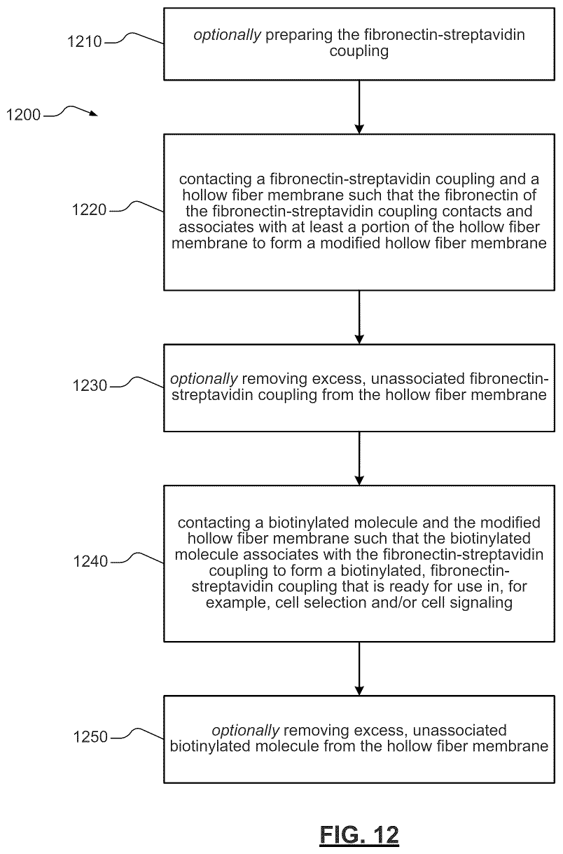

A first fluid flow path 506 may be fluidly associated with a cell growth chamber (also referred to as a “bioreactor”) 501 to form the first fluid circulation path 502. The cell growth chamber 501 may be used as the cell growth chamber 24 illustrated in FIG. 1 and/or the cell growth chamber 100 illustrated in FIG. 2. A first fluid may flow into cell growth chamber 501 through an intracapillary inlet port 501A, which may be used as an outlet in reverse. During a method of readying the hollow fiber membrane for cell expansion of natural killer and other like cells, for example, as discussed below, in the instance of FIGS. 12-14, the fibronectin-streptavidin coupling, or the components thereof, may be introduced into the hollow fiber membrane through the first fluid flow path 506 and into the intracapillary inlet port 501A. For example, the fibronectin extracellular matrix (ECM) protein, streptavidin protein, and biotinylated molecule may be introduced into the hollow fiber membrane through the first fluid flow path 506 separately and sequentially over a period of time (e.g., three days). The introduction may include one or more washout tasks to remove unbound proteins and reagents.

The first fluid may exit the cell growth chamber via an intracapillary outlet port 501B, which may be used as an inlet in reverse. For example, once in the intracapillary space 502, the fibronectin of the fibronectin-streptavidin coupling, or additionally, or alternatively, the fibronectin extracellular matrix protein separately, may contact and bind to (for example, coats) the interior-facing surface of the hollow fiber membrane and any excess unbound coupled protein, or additionally, or alternatively, the fibronectin extracellular matrix protein separately may be removed from the intracapillary space 502 through the intracapillary outlet port 501B. Similarly, once in the intracapillary space 502, the streptavidin of the fibronectin-streptavidin coupling, or additionally, or alternatively, the streptavidin protein separately, may contact and bind to (for example, coats) the interior-facing surface of the hollow fiber membrane and any excess, unbound protein, or additionally, or alternatively, the streptavidin protein separately may be removed from the intracapillary space 502 through the intracapillary outlet port 501B.

In at least one example embodiment, after a period of time, the biotinylated molecule may be introduced into the hollow fiber membrane through the first fluid flow path 506 via into the intracapillary inlet port 501A. The biotinylated molecule may include a cell-specific epitope that is selected to bind to target cells. While in the intracapillary space 502, the biotinylated molecule may bind to the fibronectin-streptavidin coupling, and more particularly, to the streptavidin, to form a biotinylated, fibronectin-streptavidin coupling that is ready for use in cell selection and/or cell signaling (including differentiation) applications. In at least one example embodiment, a washing process may be used to flow a washing media through the first fluid flow path 506, into the intracapillary inlet port 501A, through the hollow fiber membrane, and thorough the intracapillary outlet port 501B, for example, to remove any excess, unbound protein or biotinylated molecule.

With renewed reference to FIG. 8, in at least one example embodiment, the first fluid circulation path 502 may include a pressure gauge 510 configured to measure a pressure of the first fluid leaving the cell growth chamber 501. In at least one example embodiment, the first fluid circulation path 502 may include an intracapillary circulation pump 512 configured to control a first fluid flow rate. For example, the intracapillary circulation pump 512 may be configured to pump the first fluid in a first direction or a second direction that is opposite to the first direction. In the later instance, the intracapillary outlet port 501B may be used as an inlet, and the intracapillary inlet port 501A as an outlet. In at least one example embodiment, the first fluid circulation path 502 may include a sample port 516 and/or sample coil 518 configured for first fluid sample extraction. In at least one example embodiment, the first fluid circulation path 502 may include a pressure/temperature gauge 520 configured to detect the pressure and/or temperature of the first fluid during operation. In at least one example embodiment, the first fluid may enter the intracapillary loop 502 via valve 514. In at least one example embodiment, a portion of the cells may be flushed from the intracapillary loop 502 into a harvest bag 599, for example, via valve 598. It should be recognized that, in various example embodiments, the first fluid circulation path 502 may include additional or fewer valves, pressure gauges, pressure sensors, temperature sensors, ports, and/or other devices disposed to isolate and/or measure characteristics of the first fluid along portions of the intracapillary loop 502.

A second fluid may flow into cell growth chamber 501 through an extracapillary inlet port 501C. The second fluid may leave the cell growth chamber 501 via an extracapillary outlet port 501D. In at least one example embodiment, the second fluid in the extracapillary loop 504 may contact an exterior facing surface of hollow fibers disposed in the cell growth chamber 501 thereby allowing diffusion of small molecules into and out of the hollow fibers. In at least one example embodiment, the extracapillary loop 504 may include a pressure/temperature gauge 524 configured to measure a pressure and/or temperature of the second fluid before the second fluid enters the cell growth chamber 501. In at least one example embodiment, the extracapillary loop 504 may include a pressure gauge 526 that is configured to measure a pressure of the second fluid, for example, as it leaves the cell growth chamber 501. In at least one example embodiment, the extracapillary loop 504 may include a sample port 530 configured for second fluid sample extraction.

In at least one example embodiment, the extracapillary loop 504 may include an extracapillary circulation pump 528 and an oxygenator or gas transfer module 532. For example, after leaving the cell growth chamber 501, the second fluid may pass through the extracapillary circulation pump 528 and to and through the oxygenator or gas transfer module 532. In at least one example embodiment, the extracapillary circulation pump 528 may be configured to control a second fluid flow rate. For example, like the intracapillary circulation pump 512, the extracapillary circulation pump 528 may be configured to pump the second fluid in a first direction or a second direction that is opposite to the first direction. In the later instance, the extracapillary outlet port 501D may be used as inlet, and the extracapillary inlet port 501C as an outlet.

In at least one example embodiment, the second fluid flow path 522 may be fluidly associated with the oxygenator or gas transfer module 532 via an oxygenator inlet port 534 and an oxygenator outlet port 536. For example, the second fluid may flow into the oxygenator or gas transfer module 532 via the oxygenator inlet port 534 and may leave or exit the oxygenator or gas transfer module 532 via the oxygenator outlet port 536. In at least one example embodiment, the oxygenator or gas transfer module 532 may be configured to add oxygen to and/or remove bubbles from the second fluid. For example, air and/or gas may flow into the oxygenator or gas transfer module 532 via a first filter 538 and may leave or exit (i.e., flow out of) the oxygenator or gas transfer device 532 through a second filter 540. The first and second filters 538, 540 may be configured to reduce or prevent contaminants from entering the oxygenator or gas transfer module 532. The second fluid in the second fluid circulation path 504 may be in equilibrium with gas entering the oxygenator or gas transfer module 532. In at least one example embodiment, air and/or gas may be purged from the cell expansion system 500, for example, during a priming sequence, air and/or gas may be vented to the atmosphere via the oxygenator or gas transfer module 532. It should be recognized that, in at least one other example embodiment, a second fluid circulation path 504 may include additional or fewer valves, pressure gauges, pressure sensors, temperature sensors, ports, and/or other devices disposed to isolate and/or measure characteristics of the second fluid along portions of the extracapillary loop 504.

In at least one example embodiment, an air removal chamber (ARC) 556 may be fluidly associated with the first circulation path 502. The air removal chamber 556 may include one or more ultrasonic sensors. For example, the air removal chamber 556 may include upper sensor and/or lower sensor which are configured to detect air and/or a lack of fluid and/or gas-fluid interface at certain measuring positions within the air removal chamber 556. The upper sensor may be disposed near a first end (e.g., top) of the air removal chamber 556. The lower sensor may be disposed near a second end (e.g., bottom) of the air removal chamber 556. Although ultrasonic sensors are discussed, it should be appreciated that, in various example embodiments, the air removal chamber 556 may include, additionally, or alternatively, one or more other sensors, including, for example, optical sensors. Air and/or gas purged from the cell expansion system 500 during portions of a priming sequence and/or other protocols may vent to the atmosphere out air valve 560 via line 558 that may be fluidly associated with air removal chamber 556.

In at least one example embodiment, the first fluid may include cells (for example, from a first fluid container (which can also be referred to as a first media bag or a first bag) 562 and also fluid media (e.g., intracapillary media or fluid) from a second fluid container (which can also be referred to as a second media bag or a second bag) 546. Materials (i.e., cells and/or intracapillary media) form the first and second fluid containers 562, 546 may enter the first fluid circulation path 502 via a first fluid flow path 506. The first fluid container 562 may be fluidly associated with the first fluid flow path 506 and the first fluid circulation path 502 via valve 564. In at least one example embodiment, the second fluid container 546 and a third fluid container (which can also be referred to as a third media bag or third bag) 544 may be fluidly associated with the first fluid inlet path 542, for example, via valves 548 and 550, respectively, or with a second fluid inlet path 574, for example, via valves 570 and 576, respectively. In at least one example embodiment, the materials from the second fluid container 546 and/or the third fluid container 544 may be in fluid communication with a first sterile sealable input priming path 508 and/or a second sterile sealable input priming path 509.

In at least one example embodiment, a fourth fluid container (which can also be referred to as a fourth media bag or a fourth bag) 568 may include an extracapillary media, and a fifth fluid container (which can also be referred to a fifth media bag or a fifth bag) 566 may include a wash solution. Materials (i.e., extracapillary media and/or wash solution) from the fourth and fifth fluid containers 568, 566 may enter the first fluid circulation path 502 and/or the second fluid circulation path 504. For example, in at least one example embodiment, the fifth fluid container 566 may be fluidly associated with valve 570, where valve 570 is fluidly associated with first fluid circulation path 502, for example, via a distribution valve 572 and a first fluid inlet path 542. In at least one example embodiment, the fifth fluid container 566 may be fluidly associated with the second fluid circulation path 504 via the second fluid inlet path 574 and an extracapillary inlet path 584, for example, by opening valve 570 and closing distribution valve 572. The fourth fluid container 568 may be fluidly associated with valve 576, where valve 576 is fluidly associated with first fluid circulation path 502, for example, via the first fluid inlet path 542 and the distribution valve 572. In at least one example embodiment, the fourth fluid container 568 may be fluidly associated with the second fluid inlet path 574 by opening valve 576 and closing the distribution valve 572. In at least one example embodiment, the first fluid inlet path 542 and/or the second fluid inlet path 574 may be fluidly associated with an optional heat exchanger 552.

In at least one example embodiment, fluid may advance to the intracapillary loop 502 from the first fluid inlet path 542 and/or the second fluid inlet path 574 via an intracapillary inlet pump 554, and fluid may advance to the extracapillary loop 504 via an extracapillary inlet pump 578. In at least one example embodiment, an air detector 580 may also be associated with the extracapillary inlet path 584. The air detector 580 may include, for example, an ultrasonic sensor. In at least one example embodiment, the first and second fluid circulation paths 502, 504 may be fluidly associated with a waste line 588. For example, when valve 590 is in an open state or position, the intracapillary media may flow through the waste line 588 to a waste bag (also referred to as an outlet bag) 586. When valve 582 is opened, extracapillary media may flow through the waste line 588 to the waste bag 586. In at least one example embodiment, cells may be harvested, for example, via a cell harvest path 596. For example, cells from the cell growth chamber 501 may be harvested by pumping the intracapillary media containing the cells through the cell harvest path 596 and also valve 598 to a cell harvest bag 599.

In at least one example embodiment, as illustrated, the fluid in the first fluid circulation path 502 and second fluid circulation path 504 flows through cell growth chamber 501 in the same direction (i.e., a co-current configuration). Although not illustrated, it should be recognized that, in various example embodiments, the cell expansion system 500 may also be configured to flow in a counter-current conformation. As illustrated in FIG. 8, fluid in the first fluid circulation path 502 may enter the bioreactor 501 at the intracapillary inlet port 501A and may leave or exit the bioreactor 501 at the intracapillary outlet port 501B. In at least one example embodiment, the first fluid flow path 506 may be fluidly connected to the first fluid circulation path 502, for example, via connection 517. Connection 517 may be a point or location from which the fluid may flow in opposite directions, for example, based on the direction and flow rates of the intracapillary inlet pump 554 and fluid circulation pump 512. Connection 517 may include any type of fitting, coupling, fusion, pathway, and/or tubing that allows the first fluid flow path 506 to be fluidly associated with the first fluid circulation path 502. In at least one example embodiment, connection 517 may include a T-fitting or coupling and/or a Y-fitting or coupling.

In at least one example embodiment, one or more of the gauges (e.g., pressure gauge 510, pressure/temperature gauge 520, pressure/temperature gauge 524, and/or pressure gauge 526), one or more of the valves (e.g., valve 514, valves 548, valves 550, valve 560, valve 564, valve 570, valve 572, valve 576, valve 582, valve 590, valve 596, and/or valve 598), one or more of the ports (e.g., intracapillary inlet port 501A, intracapillary outlet port 501B, extracapillary inlet port 501C, extracapillary outlet port 501D, sample port 516, sample port 530, oxygenator inlet port 534, and/or an oxygenator outlet port 536), one or more of the pumps (e.g., intracapillary circulation pump 512, extracapillary circulation pump 528, intracapillary inlet pump 554, and/or extracapillary inlet pump 578), one or more of the filters (e.g., first filter 538 and/or second filter 540), one or more coils (e.g., sample coil 518), one or more modules (e.g., oxygenator or gas transfer module 532), and/or one or more other components of the cell expansion system 500 may be in electrical communication with a control system (not shown). The control system may include a plurality of nodes, which can include various hardware, firmware, and/or software configured to control and/or communicate with the mechanical, electromechanical, and electrical components of the cell expansion system 500, including for example, a controller and a memory.

The controller (which can also be referred to as a processor), can be of any type of microcontroller, microprocessor, Field Programmable Gate Array (FPGA), Application Specific Integrated Circuit (ASIC), etc. An example controller may be the NK10DN512VOK10 microcontroller, made and sold by N9P USA, Incorporated, which is a microcontroller unit with a 32-bit architecture. Other examples controllers may include, for example, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® 15-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-S™ processors, other industry-equivalent processors, and may perform computational functions using any known or future-developed standard, instruction set, libraries, and/or architecture. The memory can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller.

FIG. 9 is a schematic of another example cell expansion system 600, like the cell expansion system 100 illustrated in FIG. 1 and/or the cell expansion system 200 illustrated in FIG. 5, that illustrates example flow paths. As illustrated, the cell expansion system 600 may include a first fluid circulation path 602 (also referred to as the “intracapillary loop” or “intracapillary space” or “IC loop”) and a second fluid circulation path 604 (also referred to as the “extracapillary loop” or “extracapillary space” or “EC loop”). In at least one example embodiment, the cells may be positioned in the intracapillary space 602, while a cell culture medium may be pumped through the extracapillary space 604 to deliver nutrients to the cells via hollow fiber membrane perfusion during expansion. It should be recognized, however, in at least one other example embodiment, cells can be positioned in the extracapillary space 604, while the cell culture medium may be pumped through the intracapillary space 602 to deliver nutrients to the cells via hollow fiber membrane perfusion during expansion. In at least one other example embodiment, cells may be positioned in the intracapillary space 602 while the cell culture medium may be pumped through both the extracapillary space 604 and the intracapillary space 602. In at least one other example embodiment, cells may be positioned in the extracapillary space 604 while the cell culture medium may be pumped through both the extracapillary space 604 and the intracapillary space 602.

A first fluid flow path 606 may be fluidly associated with a cell growth chamber (also referred to as a “bioreactor”) 601 to form the first fluid circulation path 602. The cell growth chamber 601 may be used as the hollow fiber cell growth chamber 24 of the cell expansion system 24 illustrated in FIG. 1 and/or the hollow fiber cell growth chamber 100 illustrated in FIG. 1. A first fluid may flow into the cell growth chamber 601 through an intracapillary inlet port 601A. During the method of readying the hollow fiber membrane for cell expansion of natural killer and/or other like cells, for example, as discussed below, in the instance of FIGS. 12-14, the fibronectin-streptavidin coupling, or the components thereof, may be introduced into the hollow fiber membrane through the first fluid flow path 606 and into the intracapillary inlet port 601A. For example, the fibronectin extracellular matrix (ECM) protein, streptavidin protein, and biotinylated molecule may be introduced into the hollow fiber membrane through the first fluid flow path 606 separately and sequentially over a period of time (e.g., three days). The introduction may include one or more washout tasks to remove unbound proteins and reagents.

The fluid may exit the cell growth chamber 601 via an intracapillary outlet port 601B, which may be used as an inlet in reverse. For example, once in the first fluid circulation path 602, the fibronectin of the fibronectin-streptavidin coupling, or additionally, or alternatively, the fibronectin extracellular matrix protein separately, may contact and bind to (for example, coats) the interior-facing surface of the hollow fiber membrane and any excess, unbound protein, or additionally, or alternatively, the fibronectin extracellular matrix protein separately may be removed from the first fluid circulation path 602 through the intracapillary outlet port 601B. Similarly, once in the first fluid circulation path 602, the streptavidin of the fibronectin-streptavidin coupling, or additionally, or alternatively, the streptavidin protein separately, may contact and bind to (for example, coats) the interior-facing surface of the hollow fiber membrane and any excess, unbound protein, or additionally, or alternatively, the streptavidin protein separately may be removed from the first fluid circulation path 602 through the intracapillary outlet port 601B.

In at least one example embodiment, after a period of time, the biotinylated molecule may be introduced into the hollow fiber membrane through the first fluid flow path 606 and into the intracapillary inlet port 601A. The biotinylated molecule may include a cell-specific epitope that is selected to bind to target cells. While in the intracapillary space 602, the biotinylated molecule may bind to the fibronectin-streptavidin coupling, and more particularly, to the streptavidin, to form a biotinylated, fibronectin-streptavidin coupling that is ready for use in cell selection and/or cell signaling (including differentiation) applications. In at least one example embodiment, a washing process may be used to flow a washing media through the first fluid flow path 606, into the intracapillary inlet port 601A, through the hollow fiber membrane, and thorough the intracapillary outlet port 601B, for example, to remove any excess, unbound protein or biotinylated molecule.

With renewed reference to FIG. 9, in at least one example embodiment, the first fluid circulation path 602 may include a sensor 610. In at least one example embodiment, the sensor 610 may be configured to measure a pressure of media leaving cell growth chamber 601. In at least one example embodiment, the sensor 610 may be configured to measure a temperature of media leaving cell growth chamber 601. In at least one example embodiment, the sensor 610 may be configured to measure both the pressure and temperature of media leaving cell growth chamber 601. In at least one example embodiment, the media may flow through an intracapillary circulation pump 612 that is configured to control the rate of media flow. The intracapillary circulation pump 612 may be configured to pump the fluid in a first direction or second direction opposite the first direction.

In at least one example embodiment, the (first) media may enter the intracapillary loop 602 may enter through valve 614. In at least one example embodiment, samples of media may be obtained from sample coil 618 during operation. The media may then be returned to intracapillary inlet port 601A to complete fluid circulation path 602. In at least one example embodiment, cells grown/expanded in the cell growth chamber 601 may be flushed out of the cell growth chamber 601 into harvest bag 699 through a valve 698 and a line 697. Alternatively, when the valve 698 is closed, the cells may be redistributed within chamber 601 for further growth.

Fluid in second fluid circulation path 604 may enter cell growth chamber 601 via extracapillary inlet port 601C and may leave the cell growth chamber 601 via extracapillary outlet port 601D. In at least one example embodiment, the (second) media in the extracapillary loop 604 may be in contact with the outside of the hollow fibers in the cell growth chamber 601, thereby allowing diffusion of small molecules into and out of the hollow fibers that may be within chamber 601.

In at least one example embodiment, the second fluid circulation path 604 may include a sensor 624. In at least one example embodiment, the sensor 624 may be configured to measure a pressure of the media before the media enters the extracapillary space of the cell growth chamber 601. In at least one example embodiment, the sensor 624 may be configured to measure a temperature of the media before the media enters the extracapillary space of the cell growth chamber 601. In at least one example embodiment, the sensor 624 may be configured to measure a pressure and a temperature of the media before the media enters the extracapillary space of the cell growth chamber 601.

In at least one example embodiment, the second fluid circulation path 604 may include a sensor 626. In at least one example embodiment, the sensor 626 may be configured to measure a pressure of the media after the media exits the extracapillary space of the cell growth chamber 601. In at least one example embodiment, the sensor 626 may be configured to measure a temperature of the media after the media exits the extracapillary space of the cell growth chamber 601. In at least one example embodiment, the sensor 626 may be configured to measure a pressure and a temperature of the media after the media exits the extracapillary space of the cell growth chamber 601.

After leaving the extracapillary outlet port 601D of the cell growth chamber 601, fluid in the second fluid circulation path 604 may pass through an extracapillary circulation pump 628 to oxygenator or gas transfer module 632. The extracapillary circulation pump 628 may also pump the fluid in opposing directions. The second fluid flow path 622 may be fluidly associated with oxygenator or gas transfer module 632 via an inlet port 632A and an outlet port 632B of oxygenator or gas transfer module 632. In operation, fluid media may flow into the oxygenator or gas transfer module 632 via an inlet port 632A and may exit the oxygenator or gas transfer module 632 via an outlet port 632B. The oxygenator or gas transfer module 632 may ads oxygen to, and removes bubbles from, the media in the cell expansion system 600. In at least one example embodiment, media in the second fluid circulation path 604 may be in equilibrium with gas entering oxygenator or gas transfer module 632. The oxygenator or gas transfer module 632 may be any appropriately sized device useful for oxygenation or gas transfer. Air or gas flows into the oxygenator or gas transfer module 632 via filter 638 and out of oxygenator or gas transfer device 632 through filter 640. Filters 638 and 640 may reduce or prevent contamination of the oxygenator or gas transfer module 632 and associated media. Air or gas purged from the cell expansion system 600 during portions of a priming sequence may vent to the atmosphere via the oxygenator or gas transfer module 632.

Although the illustrated configurations for cell expansion system 600 shows the fluid media in the first fluid circulation path 602 and the second fluid circulation path 604 flowing through cell growth chamber 601 in the same direction (e.g., a co-current configuration). It should be recognized that, in various example embodiments, the cell expansion system 600 may also be configured to flow in a counter-current conformation.

In at least one example embodiment, media, including, for example, cells from a source such as a cell container (e.g., a bag) may be attached at an attachment point 662, and fluid media from a media source may be attached at an attachment point 646. The cells and media may be introduced into the first fluid circulation path 602 via a first fluid flow path 606. The attachment point 662 may be fluidly associated with the first fluid flow path 606 via a valve 664, and the attachment point 646 may be fluidly associated with the first fluid flow path 606 via a valve 650. A reagent source may be fluidly connected to point 644 and may be associated with a fluid inlet path 642 via valve 648 or a second fluid inlet path 674 via valves 648 and 672.

An air removal chamber (ARC) 656 may be fluidly associated with a first circulation path 602. In at least one example embodiment, the air removal chamber 656 may include one or more sensors including an upper sensor and lower sensor to detect air, a lack of fluid, and/or a gas/fluid interface (e.g., an air/fluid interface) at certain measuring positions within the air removal chamber 656. For example, ultrasonic sensors may be used near the bottom and/or near the top of the air removal chamber 656 to detect air, fluid, and/or an air/fluid interface at these locations. It should be appreciated that numerous other types of sensors may be incorporated into the cell expansion system 600 without departing from the spirit and scope of the present disclosure. For example, in at least one example embodiment, optical sensors may be used in accordance with embodiments of the present disclosure. Air or gas purged from the cell expansion system 600 during portions of the priming sequence or other protocols may vent to the atmosphere out of the air valve 660 via line 658 that may be fluidly associated with an air removal chamber 656.

An extracapillary media source may be attached to an extracapillary media attachment point 668 and/or a wash solution source may be attached to a wash solution attachment point 666 so to add extracapillary media and/or wash solution to either the first or second fluid flow path. The attachment point 666 may be fluidly associated with valve 670 that may be fluidly associated with the first fluid circulation path 602 via valve 672 and the first fluid inlet path 642. Alternatively, the attachment point 666 may be fluidly associated with the second fluid circulation path 604 via a second fluid inlet path 674 and the second fluid flow path 684 by opening valve 670 and closing valve 672. Likewise, the attachment point 668 may be fluidly associated with valve 676 that may be fluidly associated with the first fluid circulation path 602 via first fluid inlet path 642 and valve 672. Alternatively, the fluid container 668 may be fluidly associated with second fluid inlet path 674 by opening valve 676 and closing distribution valve 672.

In the intracapillary loop, fluid may be initially advanced by the intracapillary inlet pump 654. In the extracapillary loop, fluid may be initially advanced by the extracapillary inlet pump 678. An air detector 680, such as an ultrasonic sensor, may also be associated with the extracapillary inlet path 684.

In at least one embodiment, the first and second fluid circulation paths 602 and 604 may be connected to waste line 688. When valve 690 is opened, intracapillary media may flow through waste line 688 and to waste or outlet bag 686. Likewise, when valve 692 is opened, the extracapillary media may flow to waste or outlet bag 686.

After cells have been grown in cell growth chamber 601, the cells may be harvested via cell harvest path 697. Here, cells from cell growth chamber 601 may be harvested by pumping the intracapillary media containing the cells through cell harvest path 697, with valve 698 open, into cell harvest bag 699.

In at least one example embodiment, various components of the cell expansion system 600 may be contained or housed within a machine or housing, such as cell expansion machine 202, wherein the machine maintains cells and media, for example, at a predetermined temperature. In at least one example embodiments, components of the cell expansion system 600 and the cell expansion system 500 may be combined. In at least one example embodiment, a cell expansion system may include fewer or additional components than those shown in FIGS. 1 and 5 and still be within the scope of the present disclosure.

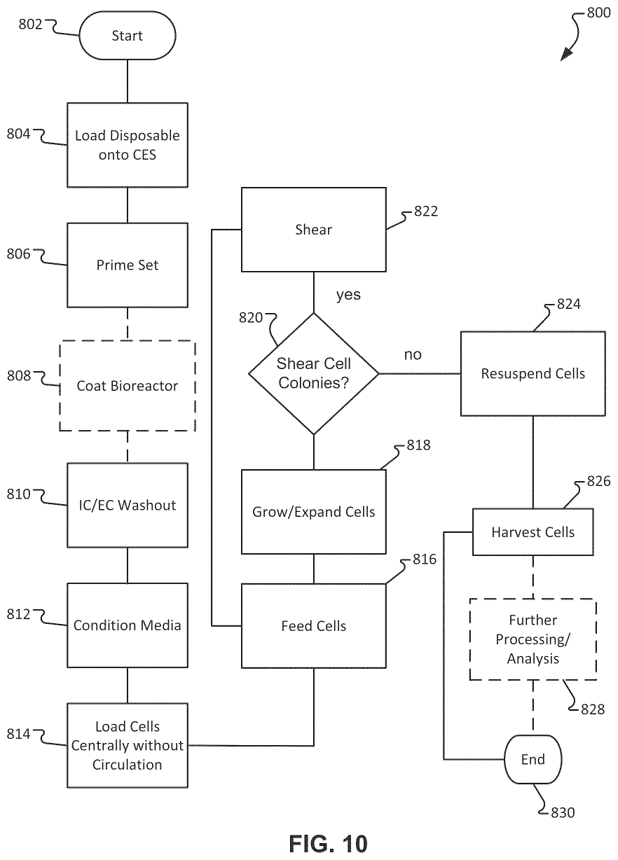

FIG. 10 illustrates an example process 800 for expanding cells that may be used with a cell expansion system, such as cell expansion system 10 illustrated in FIG. 1 and/or the cell expansion system 200 illustrated in FIG. 5 and/or the cell expansion system 500 illustrated in FIG. 8 and/or the cell expansion system 600 illustrated in FIG. 9. Once initiated 802, the process 800 may include loading a disposable tubing set 804 onto the cell expansion system and priming 806 the system. In at least one example embodiment, a user or an operator may provide instructions to the system to prime by selecting a task for priming. In at least one example embodiment, tasks for priming may be a pre-programmed task. The process 800 may then proceed to coat the cell growth chamber (which can also be referred to as a “bioreactor”) 808, in which the cell growth chamber may be optionally coated with a coating agent. The coating step 808 is shown with dashed lines to indicate that it is an optional step depending on the cell type being expanded, operator choice, or other considerations or factors.

When the process 800 includes coating step 808, a reagent may be loaded into an intracapillary loop until a reagent container is empty. The reagent may be chased from an air removal chamber into the intracapillary loop and the reagent may then be circulated in the intracapillary loop. In at least one example embodiment, a coating reagent including fibronectin may be used. Once the cell growth chamber is coated 808 (or following prime step 806 if the cell growth chamber is not coated), the process 800 may include an intracapillary/extracapillary washout task 810, where fluid on the intracapillary circulation loop and on the extracapillary circulation loop is replaced. The replacement volume may be determined by the number of intracapillary Volumes and extracapillary Volumes exchanged.

To maintain the proper or desired gas concentration across the fibers in the cell growth chamber, the condition media task 812 may be executed to allow the media to reach equilibrium with the provided gas supply before cells are loaded into the cell growth chamber. For example, rapid contact between the media and the gas supply provided by the gas transfer module or oxygenator may be provided by using a high extracapillary circulation rate. The system may then be maintained in a proper or desired state until a user or operator is ready to load cells into the cell growth chamber. In at least one example embodiment, the system may be conditioned with complete media. Complete media may be any media source used for cell growth. In at least one example embodiment, complete media may include alpha MEM (α-MEM) and/or fetal bovine serum (FBS).

The process 800 may include loading cells 814 (e.g., natural killer cells) into the cell growth chamber, for example, from a cell inlet bag. In at least one example embodiment, the cells may be loaded into the cell growth chamber from the cell inlet bag until the bag is empty. Cells may then be chased from the air removal chamber to the cell growth chamber. In embodiments that utilize larger chase volumes, cells may be spread and move toward the intracapillary outlet. In at least one example embodiment, the distribution of cells may be promoted across the membrane via intracapillary circulation, such as through an intracapillary circulation pump, with no intracapillary inlet.

In other embodiments, a “Load Cells Centrally without Circulation” task 814 may be used, in which a first volume of fluid at a first flow rate including a plurality of cells may be loaded into the cell expansion system, in which the cell expansion system includes a cell growth chamber. A second volume of fluid at a second flow rate comprising media may then be loaded into a portion of a first fluid circulation path, for example, to position the first volume of fluid in a first portion of the cell growth chamber. In at least one example embodiment, the first portion of the cell growth chamber may include about a central region of the cell growth chamber.

In at least one example embodiment, the first volume may be the same as the second volume. In at least one example embodiment, the first flow rate may be the same as the second flow rate. In at least one example embodiment, the first volume may be different from the second volume. In at least one example embodiment, the first flow rate is different from the second flow rate. In at least one example embodiment, the sum of the first volume and the second volume may equal a percentage or proportion of the volume (e.g., total volume) of the first fluid circulation path. For example, the sum of the first volume and the second volume may be about 50% of the volume (e.g., total volume) of the first fluid circulation path.

In at least one example embodiment, fluid in the first fluid circulation path flows through an intracapillary space of the cell growth chamber. In at least one example embodiment, fluid in a second fluid circulation path flows through an extracapillary space, for example, of the cell growth chamber. In at least one example embodiment, the sum of the first volume and the second volume may be about 50%, or another percentage or proportion according to embodiments, of the volume of the intracapillary loop, for example. In at least one example embodiment, the sum of the first volume and the second volume may be about 50%, or another percentage or proportion according to embodiments, of the volume of another fluid path, loop, etc., as applicable. Other percentages or proportions may be used, including, for example, any percentage between and including about 1% and about 100%.

Following the loading of the cells 814, the process 800 may include feeding the cells 816. The cells may be expanded or grown 818. While step 818 is shown after step 816, it should be recognized that in at least one example embodiment, step 818 may occur before, or simultaneous with, step 816. Next, the process 800 proceeds to query 820 to determine whether any cell colonies, microcolonies, or clusters have formed. A cell colony, micro-colony, or cluster may be a group of one or more attached cells. If a cell colony, micro-colony, or cluster has formed, process 800 proceeds “yes” to shear 822 any cell colonies, micro-colonies, or clusters. For example, after expanding a plurality of cells for a first time period, the cells may be circulated at a first circulation rate during a second time period to reduce a number of cells in a cell colony, micro-colony, or cluster. In at least one example embodiment, the circulating the cells at the first circulation rate may cause the cell colony to incur a shear stress, in which one or more cells in the cell colony may break apart from the cell colony. In at least one example embodiment, reducing the number of cells in the cell colony, micro-colony, or cluster may provide a single cell suspension, for example. In at least one example embodiment, circulating the cells to shear any colony, micro-colony, or cluster 822 may be used every one or two days, for example, during cell culture to maintain uniform cell density and nutrient diffusion. In at least one example embodiment, such shearing of any micro-colonies, colonies, or clusters may begin on or after Day Four, for example. Other days or time periods on which to begin such shearing may be used according to other example embodiments. Following shearing 822, the process 800 may next return to feed cells 816.

If it is determined at query 820 not to shear any cell colonies or clusters, or if none exist, for example, the process 800 proceeds “no” to resuspend cells 824. In at least one example embodiment, circulating the cells may be used to uniformly resuspend those cells that may be loosely adhered during culture. In at least one example embodiment, step 824 may include circulating the cells to uniformly resuspend those cells that may be loosely adhered prior to initiating a harvest task, or other task to remove cells from the cell growth chamber. Following the resuspension of the cells 824, the process 800 may next proceed to harvesting the cells 826. Further processing of the removed cells or other analysis may optionally be performed at step 828, and the process 800 may then terminate at END operation 830. If it is not desired to perform further processing/analysis, the process 800 terminates at END operation 830.

It should be appreciated that the operational steps depicted are offered for purposes of illustration and may be rearranged, combined into other steps, used in parallel with other steps, etc., according to embodiments of the present disclosure. Fewer or additional steps may be used in embodiments without departing from the spirit and scope of the present disclosure. Also, steps (and any sub-steps), such as priming, coating the cell growth chamber, loading cells, for example, may be performed automatically in some embodiments, such as by a processor executing pre-programmed tasks stored in memory, in which such steps are provided merely for illustrative purposes.

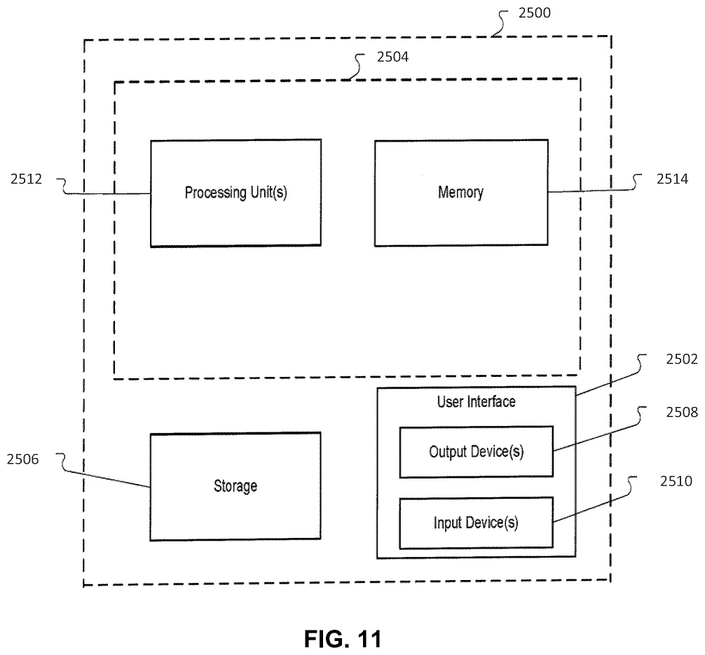

FIG. 11 illustrates example components of a computing system 2500 upon which embodiments of the present disclosure may be implemented. Computing system 2500 may be used in embodiments, for example, where a cell expansion system (such as cell expansion system 10 illustrated in FIG. 1 and/or the cell expansion system 200 illustrated in FIG. 5 and/or the cell expansion system 500 illustrated in FIG. 8 and/or the cell expansion system 600 illustrated in FIG. 9) uses a processor to execute tasks, such as custom tasks or pre-programmed tasks performed as part of processes such as processes illustrated and/or described herein. In certain variations, pre-programmed tasks may include, follow “Ready Membrane”, “IC/EC Washout” and/or “Feed Cells,” for example.

As illustrated, the computing system 2500 may include a user interface 2502, a processing system 2504, and/or storage 2506. The user interface 2502 may include output device(s) 2508 and/or input device(s) 2510. The output device(s) 2508 may include one or more touch screens, in which the touch screen may include a display area for providing one or more application windows. The touch screen may also be an input device 2510 that may receive and/or capture physical touch events from a user or operator, for example. The touch screen may comprise a liquid crystal display (LCD) having a capacitance structure that allows the processing system 2504 to deduce the location(s) of touch event(s). The processing system 2504 may then map the location of touch events to UI elements rendered in predetermined locations of an application window. The touch screen may also be configured to receive touch events through one or more other electronic structures, according to embodiments. Other output devices 2508 may include a printer, speaker, etc. Other input devices 2510 may include a keyboard, other touch input devices, mouse, voice input device, etc.

Processing system 2504 may include a processing unit 2512 and/or a memory 2514. In at least one example embodiment, the processing unit 2512 may be a general-purpose processor operable to execute instructions stored in memory 2514. Processing unit 2512 may include a single processor or multiple processors. Further, each processor may be a multi-core processor having one or more cores to read and execute separate instructions. The processors may include general purpose processors, application specific integrated circuits (ASICs), field programmable gate arrays (FPGAs), other integrated circuits, etc.

The memory 2514 may include any short-term or long-term storage for data and/or processor executable instructions, according to embodiments. The memory 1014 may include, for example, Random Access Memory (RAM), Read-Only Memory (ROM), or Electrically Erasable Programmable Read-Only Memory (EEPROM). Other storage media may include, for example, CD-ROM, tape, digital versatile disks (DVD) or other optical storage, tape, magnetic disk storage, magnetic tape, other magnetic storage devices, etc.

Storage 2506 may be any long-term data storage device or component. Storage 2506 may include one or more of the systems described in conjunction with the memory 2514, according to embodiments. The storage 2506 may be permanent or removable. Storage 2506 may be configured to store data generated or provided by the processing system 2504.

In order for cells (such as natural killer cells (also referred to as NK cells)) to better adhere to the hollow fibers defining a cell growth chamber (e.g., cell growth chamber 24 illustrated in FIG. 1 and/or cell growth chamber 100 illustrated in FIG. 3 and/or cell growth chamber 501 illustrated in FIG. 8 and/or cell growth chamber 601 illustrated in FIG. 9) for cell expansion, differentiation, harvesting, and/or activation it may be beneficial to modify the surface of the hollow fiber (for example, an interior-facing surface (or hollow fiber membrane lumen)) when cells are expanded in the intracapillary space (e.g., intracapillary space 130) or the exterior-facing surface when cells are expanded in the extracapillary space (e.g., extracapillary space 110) in some way. For example, fibronectin (FN) and/or collagen can be used as surface modifiers and/or the hollow fibers can be exposed to radiation. Natural killer cells, for example, however, do not readily bind to fibronectin or collagen and/or gamma-treated surfaces.

In various aspects, the present disclosure provides methods and materials for binding adherent and non-adherent cell types (i.e., lymphocytes, hematopoietic stem cells, hematopoietic progenitor cells, endothelial cells, mesenchymal stromal cells, or any combination thereof) to hollow fiber membranes. For example, the present disclosure provides methods and materials for binding of natural killer cells (isolated, for example, buffy coated blood products, leukopaks, and/or cord blood) and/or other like cells to hollow fiber membranes.

The methods may include, for example, using a biotinylated molecule selected for target cell affinity and an extracellular matrix component to be associated with both the hollow fiber membrane defining the cell growth chamber and the extracellular matrix. The biotinylated molecule may support, for example, the stimulation and/or monoculture of the natural killer and/or other similar cells using automated, perfusion-based cell expansion system. In at least one example embodiment, the biotinylated molecule may include cytokine, epitope, ligand, monoclonal antibody, stain, aptamer, chemokine, biotin, or any combination thereof.

In at least one example embodiment, the extracellular matrix component may be selected from the group consisting of fibronectin, vitronectin, fibrinogen, collagen, laminin, truncations thereof, and combinations thereof. In at least one example embodiment, the extracellular matrix component may include an extracellular matrix component-streptavidin coupling, where the extracellular matrix component of the extracellular matrix component-streptavidin coupling associates with the surface of the hollow-fiber membrane and the streptavidin of the extracellular matrix component-streptavidin coupling associates with the biotinylated molecule. In at least one example embodiment, the hollow fiber member may include a mass ratio of the streptavidin to the biotinylated molecule of greater than or equal to about 1:3 to less than or equal to about 1:9. When the extracellular matrix component-streptavidin coupling includes a fibronectin-streptavidin coupling, the fibronectin may have a molecular weight greater than or equal to about 440 kDa to less than or equal to about 500 kDa and the streptavidin may have a molecular weight greater than or equal to about 53 kDa to less than or equal to about 55 kDa.

The methods may include, for example, preparing the extracellular matrix component-streptavidin coupling. In at least one example embodiment, preparing the extracellular matrix component-streptavidin coupling may include covalently coupling the extracellular matrix component and the streptavidin. For example, preparing a fibronectin-streptavidin coupling may include reconstituting lyophilized fibronectin with streptavidin by immerging the lyophilized fibronectin and streptavidin in water. In at least one example embodiment, preparing the extracellular matrix component-streptavidin coupling may forming hydrogen bonds between the extracellular matrix component and the streptavidin. In at least one example embodiment, preparing the extracellular matrix component-streptavidin coupling may include introducing the extracellular matrix component and streptavidin sequentially into a cell growth chamber during which the extracellular matrix component and streptavidin are associated by both classical and non-classical hydrogen bonding with no necessitated covalent linkage.

The methods may include, for example, when the extracellular matrix component-streptavidin coupling is pre-formed, contacting the extracellular matrix component-streptavidin coupling to a surface of the hollow-fiber membrane. In at least one example embodiment, the contacting may include introducing the extracellular matrix component-streptavidin coupling into an intracapillary space of the hollow fiber membrane where the extracellular matrix component of the extracellular matrix component-streptavidin coupling contacts and adheres to (for example, coats) at least a portion of an interior-facing surface of the hollow fiber membrane. In at least one example embodiment, the contacting may include introducing the extracellular matrix component-streptavidin coupling into an extracapillary space of the hollow fiber membrane where the extracellular matrix component of the extracellular matrix component-streptavidin coupling contacts and adheres to (for example, coats) at least a portion of an exterior-facing surface of the hollow fiber membrane. In at least one example embodiment, the contacting may include introducing the extracellular matrix component-streptavidin coupling into both an intracapillary space and an extracapillary space of the hollow fiber membrane where the extracellular matrix component of the extracellular matrix component-streptavidin coupling contacts and adheres to at least a portion of an interior-facing surface and at least a portion of an exterior-facing surface of the hollow fiber membrane. The methods may include, for example, after the contacting of the extracellular matrix component-streptavidin coupling and the surface of the hollow fiber membrane, and before the contacting of the biotinylated molecule and the hollow fiber membrane, washing the hollow fiber to remove any unbound and excess portions of the extracellular matrix component-streptavidin coupling.

The methods may include, for example, when the extracellular matrix component and streptavidin are introduced sequentially, first contacting the extracellular matrix component and a surface of the hollow fiber membrane and then contacting the streptavidin and the surface of the hollow-fiber membrane. In at least one example embodiment, the contacting may include introducing the extracellular matrix component and subsequently the streptavidin into an intracapillary space of the hollow fiber membrane where the extracellular matrix component contacts and adheres to (for example, coats) at least a portion of an interior-facing surface of the hollow fiber membrane and the streptavidin contacts and adheres to the extracellular matrix component via both classical and non-classical hydrogen bonding. In at least one example embodiment, the contacting may include introducing the extracellular matrix component and subsequently the streptavidin into an extracapillary space of the hollow fiber membrane where the extracellular matrix component contacts and adheres to (for example, coats) at least a portion of an exterior-facing surface of the hollow fiber membrane and the streptavidin contacts and adheres to the extracellular matrix component via both classical and non-classical hydrogen bonding. In at least one example embodiment, the contacting may include introducing the extracellular matrix component and subsequently the streptavidin into both an intracapillary space and an extracapillary space of the hollow fiber membrane where the extracellular matrix component contacts and adheres to at least a portion of an interior-facing surface and at least a portion of an exterior-facing surface of the hollow fiber membrane and the streptavidin contacts and adheres to the extracellular matrix component via both classical and non-classical hydrogen bonding. The methods may include, for example, after the contacting of the extracellular matrix component and the surface of the hollow fiber membrane, and before the contacting of the streptavidin and the hollow fiber membrane, washing the hollow fiber to remove any unbound and excess portions of the extracellular matrix component. The methods may include, for example, after the contacting of the streptavidin and the surface of the hollow fiber membrane, and before the contacting of the biotinylated molecule and the hollow fiber membrane, washing the hollow fiber to remove any unbound and excess portions of the streptavidin.

The methods may include, for example, contacting the biotinylated molecule and the surface of the hollow-fiber membrane where the biotinylated molecule binds to the extracellular matrix component or the extracellular matrix component-streptavidin coupling. In at least one example embodiment, for example, when the extracellular matrix component or the extracellular matrix component-streptavidin coupling is introduced into the intracapillary space of the hollow fiber membrane, the contacting may include introducing the biotinylated molecule into the intracapillary space. In at least one example embodiment, for example, when the extracellular matrix component or the extracellular matrix component-streptavidin coupling is introduced into the extracapillary space of the hollow fiber membrane, the contacting may include introducing the biotinylated molecule into the extracapillary space. In at least one example embodiment, for example, when the extracellular matrix component or the extracellular matrix component-streptavidin coupling is introduced into the intracapillary space and the extracapillary space of the hollow fiber membrane, the contacting may include introducing the biotinylated molecule into the intracapillary space and the extracapillary space. The methods may include, for example, after the contacting of the biotinylated molecule and the surface of the hollow fiber membrane, washing the hollow fiber to remove any unbound and excess portions of the biotinylated molecule.

FIG. 12 is a flowchart illustrating an example method 1200 for readying a cell growth chamber (e.g., cell growth chamber 24 illustrated in FIG. 1 and/or cell growth chamber 100 illustrated in FIG. 3 and/or cell growth chamber 501 illustrated in FIG. 8 and/or cell growth chamber 601 illustrated in FIG. 9) for cell expansion, differentiation, harvesting, and/or activation, for example, of natural killer cells and/or other similar cells, using a biotinylated protein coupling, such as a fibronectin-streptavidin (FN-SN) coupling. The method 1200 may include contacting 1220 a fibronectin-streptavidin coupling and the hollow fiber membrane during which the fibronectin of the fibronectin-streptavidin coupling contacts and adheres to (for example, coats) at least a portion of the hollow fiber membrane that defines the cell growth chamber and forms a modified hollow fiber membrane.

In at least one example embodiment, the contacting 1220 may include introducing the fibronectin-streptavidin coupling into an intracapillary space of the hollow fiber membrane where the fibronectin of the fibronectin-streptavidin coupling contacts and adheres to (for example, coats) at least a portion of an interior-facing surface of the hollow fiber membrane. In at least one example embodiment, the contacting 1220 may include introducing the fibronectin-streptavidin coupling into an extracapillary space of the hollow fiber membrane where the fibronectin of the fibronectin-streptavidin coupling contacts and adheres to (for example, coats) at least a portion of an exterior-facing surface of the hollow fiber membrane. In at least one example embodiment, the contacting 1220 may including introducing the fibronectin-streptavidin coupling into both an intracapillary space and an extracapillary space of the hollow fiber membrane where the fibronectin of the fibronectin-streptavidin coupling contacts and adheres to at least a portion of an interior-facing surface and at least a portion of an exterior-facing surface of the hollow fiber membrane. In at least one example embodiment, the coating layer may coat and/or occlude pores defining the interior-facing surface of the hollow fiber membrane.

In each instance, the fibronectin of the fibronectin-streptavidin coupling may adhere to the hollow fiber membrane to form a coating layer. In at least one example embodiment, the coating layer may be a continuous coating that covers, for example, about 100% of a total surface area of the interior-facing surface of the hollow fiber membrane and/or the exterior-facing surface of the hollow fiber membrane depending upon the introduction point for the fibronectin-streptavidin coupling. In at least one example embodiment, the coating layer may be a discontinuous coating that includes, for example, one or more patterns. In at least one example embodiment, the coating layer may be a variable thickness coating that includes, for example, one or more first regions having a first thickness and one or more second regions having a second thickness that is different than the first thickness. The variable thickness coating may be a continuous or discontinuous coating.

The hollow fiber membrane has a net negative charge under physiological pH, which is greater than or equal to about 7.2 to less than or equal to about 7.4. The fibronectin has a net positive charge and adheres to the hollow fiber membrane via polarity and hydrogen bonding (including both classical and non-classical hydrogen bonding). Further, fibronectin has a naturally adhesive nature as a result of its glycoprotein structure and specific domains, which allows the fibronectin to associate with both the hollow fiber membrane (e.g., polyethersulfones (PES)) and cell membrane integrins. Although fibronectin is discussed, it should be appreciated that other extracellular matrix (ECM) proteins having net position charge (including, for example, vitronectin and/or fibrinogen and/or collagen and/or laminin, as well as their isoforms) may form couplings with streptavidin (for example, by covalent or hydrogen bonding) and adhere to one or more portions or regions of the hollow fiber membrane.

With renewed reference to FIG. 12, the fibronectin-streptavidin coupling may be contacted 1200 with the hollow fiber membrane for a first period. The first period may be greater than or equal to about 4 hours to less than or equal to about 24 hours (e.g., about 12 hours). After contacting 1200 the fibronectin-streptavidin coupling with the hollow fiber membrane, the method 1200 may further include contacting 1240 a biotinylated molecule and the modified hollow fiber membrane. In at least one example embodiment, the biotinylated molecule may be contacted 1240 with the modified hollow fiber membrane using a “Coat Bioreactor” setting of the cell expansion system.

In at least one example embodiment, for example, when the fibronectin-streptavidin coupling is introduced into the intracapillary space of the hollow fiber membrane, the contacting 1240 may include introducing the biotinylated molecule into the intracapillary space. In at least one example embodiment, for example, when the fibronectin-streptavidin coupling is introduced into the extracapillary space of the hollow fiber membrane, the contacting 1240 may include introducing the biotinylated molecule into the extracapillary space. In at least one example embodiment, for example, when the fibronectin-streptavidin coupling is introduced into the intracapillary space and the extracapillary space of the hollow fiber membrane, the contacting 1240 may include introducing the biotinylated molecule into the intracapillary space and the extracapillary space.

In each instance, the biotinylated molecule may be selected from the group consisting of: cytokine (including an interleukin or growth factor), epitope, ligand, monoclonal antibody, stains, aptamer, chemokine, biotin, and combinations thereof. The biotinylated molecule may associate with to the fibronectin-streptavidin coupling, and more particularly, to the streptavidin, to form a biotinylated, fibronectin-streptavidin coupling that is ready for use in cell selection and/or cell signaling (including differentiation) applications. In at least one example embodiment, streptavidin (having, for example, a molecular weight between about 52 kDa and about 55 kDa) may bind up to four molecules of biotin (having, for example, a molecular weight of about 244 Dalton) with a high degree of specificity and affinity (e.g., Kd=1E-14 to 1E-15) primarily through hydrogen bonding and van der Waals forces with amino acid residues which stabilize the multimeric streptavidin molecule.

In at least one example embodiment, the method 1200 may include, prior to contacting 1240 the biotinylated molecule and the modified hollow fiber membrane, removing 1230 any excess, unbound portion of the protein from the hollow fiber membrane. For example, the excess, unbound portion of the protein may be removed using a washing process. In at least one example embodiment, during the washing process, a predetermined or selected amount (e.g., about 450 milliliters) of a buffer (e.g., phosphate-buffered saline (PBS)) may be contacted with the hollow fiber membrane to remove unbound biotinylated protein and/or other biotinylated molecules prior to contacting 1240 the biotinylated molecule and the modified hollow fiber membrane.

In at least one example embodiment, the method 1200 may include, after the contacting 1240 of the biotinylated molecule and the modified hollow fiber membrane, removing 1250 any excess, unbound portion of the biotinylated molecule from the hollow fiber membrane. For example, the excess, unbound portion of the biotinylated molecule may be removed using a washing process.

In at least one example embodiment, the method 1200 may include, prior to contacting 1220 the fibronectin-streptavidin coupling and the hollow fiber membrane, preparing 1210 the fibronectin-streptavidin coupling.

In at least one example embodiment, preparing 1210 the fibronectin-streptavidin coupling may include reconstituting lyophilized fibronectin (having, for example, a molecular weight greater than or equal to about 440 kDa to less than or equal to about 500 kDa) with streptavidin (having, for example, a molecular weight greater than or equal to about 53 kDa to less than or equal to about 55 kDa) by contacting (e.g., immerging) the lyophilized fibronectin and streptavidin with deionized (DI) water (e.g., sterile deionized water (5 mg/10 mL)) at ambient temperature (e.g., greater than or equal to about 20° C. to less than or equal to about 22° C.) and subsequently diluting the admixture with phosphate-buffered saline (PBS) (e.g., about 90 mL of phosphate-buffered saline (PBS) that is free of calcium ions and magnesium ions).

In at least one example embodiment, preparing 1210 the fibronectin-streptavidin coupling may include a covalent coupling process that uses linkage modifiers and quencher chemistry to generate covalent linkages between the fibronectin and the streptavidin. For example, in at least one example embodiment, the covalent coupling process may include using a Bio-Rad LYNX Rapid Streptavidin Conjugation Kit. The covalent coupling process may occur over a period greater than or equal to about 3 hours to less than or equal to about 15 hours. In each variation, the fibronectin-streptavidin coupling may have a mass ratio of the fibronectin to the streptavidin greater than or equal to about 1:2 to less than or equal to about 1:9, and in certain aspects, optionally about 1:3.3. The selection of the specific mass ratio may be important for maintaining the functionality of the fibronectin and streptavidin during cell selection and expansion.

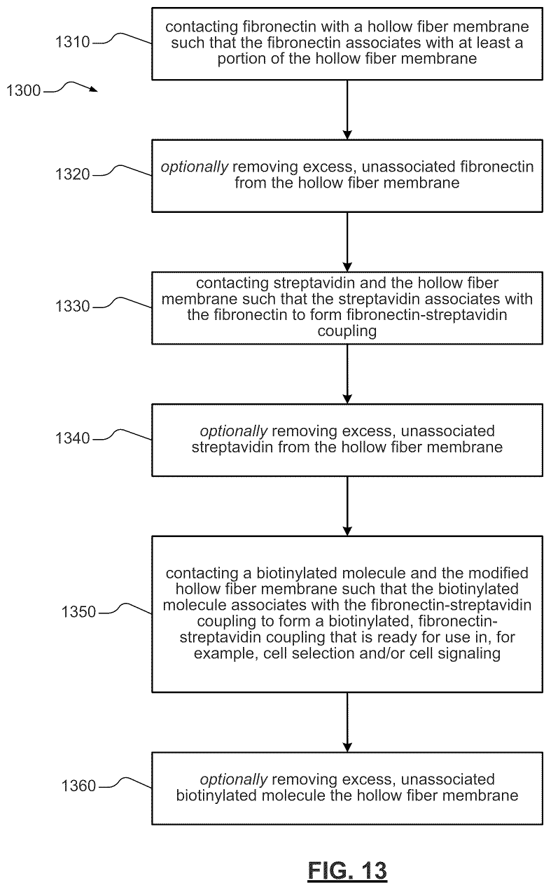

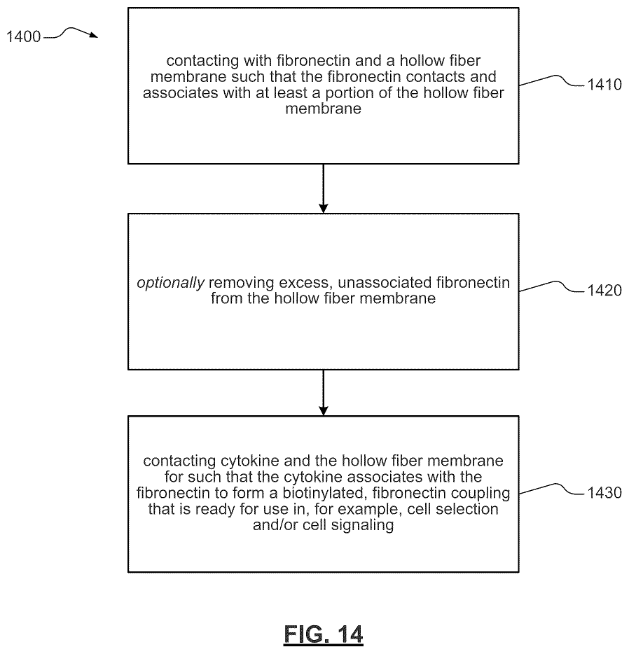

FIG. 13 is a flowchart illustrating another example method 1300 for readying a cell growth chamber (e.g., cell growth chamber 24 illustrated in FIG. 1 and/or cell growth chamber 100 illustrated in FIG. 3 and/or cell growth chamber 501 illustrated in FIG. 8 and/or cell growth chamber 601 illustrated in FIG. 9) for cell expansion, differentiation, harvesting, and/or activation, for example, of natural killer cells and/or other similar cells, using a biotinylated protein coupling, such as a fibronectin-streptavidin coupling.

The method 1300 may include contacting 1310 fibronectin (and/or other extracellular matrix proteins) and the hollow fiber membrane such that the fibronectin adheres to (for example, coats) at least a portion of the hollow fiber membrane that defines the cell growth chamber. For example, when the hollow fiber membrane includes, or is defined by, polyethersulfones (PES), during the contacting 1310 the fibronectin (and/or other extracellular matrix proteins) may adsorb to the hollow fiber membrane through ionic bonding between the net positively charged fibronectin (and/or other extracellular matrix proteins) and the negatively charged sulfone group of the hollow fiber membrane. The hollow fiber membrane may exhibit a net negative charge under physiological operating conditions (e.g., pH ranging from about 7.2 to about 7.4). In at least one example embodiment, the glycosylated portion of fibronectin may provide further adhesion to the hollow fiber membrane.

In at least one example embodiment, the contacting 1310 may include introducing the fibronectin into an intracapillary space of the hollow fiber membrane where the fibronectin contacts and adheres to (for example, coats) at least a portion of an interior-facing surface of the hollow fiber membrane. In at least one example embodiment, the contacting 1310 may include introducing the fibronectin into an extracapillary space of the hollow fiber membrane where the fibronectin contacts and adheres to (for example, coats) at least a portion of an exterior-facing surface of the hollow fiber membrane. In at least one example embodiment, the contacting 1310 may including introducing the fibronectin into both an intracapillary space and an extracapillary space of the hollow fiber membrane where the fibronectin contacts and adheres to at least a portion of an interior-facing surface and at least a portion of an exterior-facing surface of the hollow fiber membrane. In at least one example embodiment, the fibronectin coating layer may coat and/or occlude pores defining the interior-facing surface of the hollow fiber membrane.

In each instance, the fibronectin may adhere to the hollow fiber membrane to form a coating layer. In at least one example embodiment, the fibronectin coating layer may be a continuous coating that covers, for example, about 100% of a total surface area of the interior-facing surface of the hollow fiber membrane and/or the exterior-facing surface of the hollow fiber membrane depending upon the introduction point for the fibronectin. In at least one example embodiment, the fibronectin coating layer may be a discontinuous coating that includes, for example, one or more patterns. In at least one example embodiment, the fibronectin coating layer may be a variable thickness coating that includes, for example, one or more first regions having a first thickness and one or more second regions having a second thickness that is different than the first thickness. The variable thickness coating may be a continuous or discontinuous coating.

The fibronectin may be contacted 1310 with the hollow fiber membrane for a first period. The first period may be greater than or equal to about 15 hours to less than or equal to about 24 hours. After contacting 1310 the fibronectin and the hollow fiber membrane, the method 1300 may further include contacting 1330 streptavidin and the hollow fiber membrane such that the streptavidin associates with the fibronectin by hydrogen bonding to form fibronectin-streptavidin couplings. More particularly, it is believed that the net positively-charged, dimeric fibronectin composed of positively-charged lysine, arginine, and histidine residues that couple to the negatively-charged streptavidin composed of negatively-charged aspartic acid and glutamic acid residues.

In at least one example embodiment, for example, when the fibronectin is introduced into the intracapillary space of the hollow fiber membrane, the contacting 1330 may include introducing the streptavidin into the intracapillary space. In at least one example embodiment, for example, when the fibronectin is introduced into the extracapillary space of the hollow fiber membrane, the contacting 1330 may include introducing the streptavidin into the extracapillary space. In at least one example embodiment, for example, when the fibronectin is introduced into the intracapillary space and the extracapillary space of the hollow fiber membrane, the contacting 1330 may include introducing the streptavidin into the intracapillary space and the extracapillary space.

The streptavidin may be contacted 1330 with the hollow fiber membrane for a second period. The second period may be greater than or equal to about 15 hours to less than or equal to about 24 hours. After contacting 1330 the streptavidin and the hollow fiber membrane, the method 1300 may further include contacting 1350 a biotinylated molecule and the modified hollow fiber membrane. In at least one example embodiment, the biotinylated molecule may be contacted 1350 with the modified hollow fiber membrane using a “Coat Bioreactor” setting of the cell expansion system.

In at least one example embodiment, for example, when the fibronectin and streptavidin are introduced into the intracapillary space of the hollow fiber membrane, the contacting 1350 may include introducing the biotinylated molecule into the intracapillary space. In at least one example embodiment, for example, when the fibronectin and streptavidin are introduced into the extracapillary space of the hollow fiber membrane, the contacting 1350 may include introducing the biotinylated molecule into the extracapillary space. In at least one example embodiment, for example, when the fibronectin and streptavidin are introduced into the intracapillary space and the extracapillary space of the hollow fiber membrane, the contacting 1350 may include introducing the biotinylated molecule into the intracapillary space and the extracapillary space.

In at least one example embodiment, the biotinylated molecule and hollow fiber membrane may be contacted 1350 for a third period. The third period may be greater than or equal to about 15 hours to less than or equal to about 24 hours. After contacting 1350 the biotinylated molecule and the hollow fiber membrane, in at least one example embodiment, the method 1300 may further include removing 1360 any excess, unbound portion of the biotinylated molecule from the hollow fiber membrane. For example, the excess, unbound portion of the biotinylated molecule may be removed using a washing process.

In at least one example embodiment, after contacting 1310 the fibronectin and the hollow fiber membrane and before contacting 1330 the streptavidin and the hollow fiber membrane, the method 1300 may further include removing 1320 any excess, unbound portion of the fibronectin from the hollow fiber membrane. For example, the excess, unbound portion of the fibronectin may be removed using a washing process.

In at least one example embodiment, after the contacting 1330 of the streptavidin and the hollow fiber membrane and before the contacting 1350 of the biotinylated molecule and the hollow fiber membrane, the method 1300 may further include removing 1340 any excess, unbound portion of the streptavidin from the hollow fiber membrane. For example, the excess, unbound portion of the fibronectin may be removed using a washing process.

FIG. 14 is a flowchart illustrating an example method 1400 for readying a cell growth chamber (e.g., cell growth chamber 24 illustrated in FIG. 1 and/or cell growth chamber 100 illustrated in FIG. 3 and/or cell growth chamber 501 illustrated in FIG. 8 and/or cell growth chamber 601 illustrated in FIG. 9) for cell expansion, differentiation, harvesting, and/or activation, for example, of natural killer cells and/or other similar cells, using a cytokine. As illustrated, the method 1400 may include contacting 1410 fibronectin (and/or other extracellular matrix proteins) and the hollow fiber membrane during which the fibronectin contacts and adheres to (for example, coats) at least a portion of the hollow fiber membrane.

In at least one example embodiment, the contacting 1410 may include introducing the fibronectin into an intracapillary space of the hollow fiber membrane where the fibronectin contacts and adheres to (for example, coats) at least a portion of an interior-facing surface of the hollow fiber membrane. In at least one example embodiment, the contacting 1410 may include introducing the fibronectin into an extracapillary space of the hollow fiber membrane where the fibronectin contacts and adheres to (for example, coats) at least a portion of an exterior-facing surface of the hollow fiber membrane. In at least one example embodiment, the contacting 1410 may including introducing the fibronectin into both an intracapillary space and an extracapillary space of the hollow fiber membrane where the fibronectin contacts and adheres to at least a portion of an interior-facing surface and at least a portion of an exterior-facing surface of the hollow fiber membrane.

In each instance, the fibronectin may adhere to the hollow fiber membrane to form a coating layer. In at least one example embodiment, the fibronectin coating layer may be a continuous coating that covers, for example, about 100% of a total surface area of the interior-facing surface of the hollow fiber membrane and/or the exterior-facing surface of the hollow fiber membrane depending upon the introduction point for the fibronectin. In at least one example embodiment, the fibronectin coating layer may be a discontinuous coating that includes, for example, one or more patterns. In at least one example embodiment, the fibronectin coating layer may be a variable thickness coating that includes, for example, one or more first regions having a first thickness and one or more second regions having a second thickness that is different than the first thickness. The variable thickness coating may be a continuous or discontinuous coating.

The fibronectin may be contacted 1410 with the hollow fiber membrane for a first period. The first period may be greater than or equal to about 4 hours to less than or equal to about 24 hours (e.g., about 12 hours). After contacting 1410 the fibronectin and the hollow fiber membrane, the method 1400 may further include contacting 1430 a cytokine and the hollow fiber membrane. The cytokine may associate with the fibronectin to ready the hollow fiber membrane for use in cell selection and/or cell signaling (including expansion, differentiation, harvesting, and/or activation) applications. The cytokine may include a class-I cytokine and class-I cytokines may include, for example, interleukin-21 (IL-21), interleukin-2 (IL-2), interleukin-12 (IL-12), interleukin-15 (IL-15), interleukin-18 (IL-18), CD16, NKG2C/CD94, NKG2D, DNAM-1, 2B4 (CD48), Nkp30, and/or the like. The cytokine may be selected to enhance both ex vivo expansion and cytotoxicity of natural killer cells.

In at least one example embodiment, for example, when the fibronectin is introduced into the intracapillary space of the hollow fiber membrane, the contacting 1430 may include introducing the cytokine into the intracapillary space. In at least one example embodiment, for example, when the fibronectin is introduced into the extracapillary space of the hollow fiber membrane, the contacting 1430 may include introducing the cytokine into the extracapillary space. In at least one example embodiment, for example, when the fibronectin is introduced into the intracapillary space and the extracapillary space of the hollow fiber membrane, the contacting 1430 may include introducing the cytokine into the intracapillary space and the extracapillary space.

In at least one example embodiment, the method 1400 may include, after the contacting 1420 of the fibronectin and the hollow fiber member and prior to contacting 1430 the cytokine and the hollow fiber membrane, removing 1420 an excess, unbound portion of the fibronectin from the hollow fiber membrane. For example, the excess, unbound portion of the fibronectin may be removed using a washing process. In at least one example embodiment, during the washing process, about 450 mL of phosphate-buffered saline (PBS), or other buffer, may be contacted with the hollow fiber membrane to remove unbound fibronectin and/or other molecules prior to contacting 1430 the cytokine and the modified hollow fiber membrane.

Certain features of the current technology are further illustrated in the following non-limiting examples.

### Example 1

### Streptavidin Coating Preparation

In certain variations, a streptavidin coating may be prepared by contacting streptavidin to water (H₂O) to form a 10 μg/mL solution. The solution (e.g., 100 μL) may be added into wells and left to dry. After a period of time, a washing process may be applied. For example, 0.1 BSA may be washed and blocked for about 1 hour at ambient or room temperature (e.g., greater than or equal to about 20° C. to less than or equal to about 22° C.) so as to prepare the hollow fibers.

In certain variations, the streptavidin coating process may include, at Day Three, coating a polyethersulfones (PES) membrane with a phosphate-buffered saline (PBS)-fibronectin solution overnight at 37° C. by introducing the solution at 0.1-20 mL/min and circulated at greater than or equal to about 0 mL/min to less than or equal to about 20 mL/min in the intracapillary loop of the Quantum CES or similar platform. This may be followed by a phosphate-buffered saline (PBS) wash step so as to remove unbound fibronectin using, for example, the Quantum CES IC Rapid Washout or IC/EC Exchange Task. At Day Two, the wash step may be followed by the addition of phosphate-buffered saline (PBS)-streptavidin solution, which may adhere to the underlying fibronectin layer and may be circulated overnight in similar fashion and is followed by Quantum Washout Task. At Day One, the phosphate-buffered saline (PBS)-biotinylated IL-21 cytokine solution may be added and circulated at 37° C. within the intracapillary loop in a similar fashion and followed by a Quantum Washout Task prior to cell seeding.

### Example 2

### Bioreactor/Column Coating

There are several approaches for the creation of fibronectin (FN)-streptavidin (SN) foundations for the attachment of biotinylated molecules to functionalize the surface of the Quantum® System polyethersulfones (PES) hollow fiber membrane (HFM) bioreactor or preparatory columns for cell selection. For example, in certain variations, fibronectin may bind to the polyethersulfone hollow fiber membrane in the Quantum® Cell Expansion System (System) bioreactor through the adherence and expansion of adherent cells such as mesenchymal stromal/stem cells (MSCs), fibroblasts, and/or aortic endothelial cells. This process may be based on the established high affinity of streptavidin binding for biotin. While considering the available protein coupling biochemistries, it may be important to keep the protocols direct and efficient with minimal residue or reactants in order to accommodate their adaption in the manufacturing of cell therapy products. Mixing and/or linking fibronectin-streptavidin mixture or coupling may support the functionalization of the hollow fiber membrane bioreactor or column with biotinylating cytokines, chemokines, and other ligands to facilitate cell selection and expansion. Other affinity separations of biomolecules may be anticipated. In short, this protein-protein coupling may be viewed as a platform for affinity processes associated with cell therapy which uses available technology.

Two example approaches are described: (1) a simple mixture of fibronectin and streptavidin and (2) rapid covalent coupling of fibronectin and streptavidin using a Bio-Rad LYNX Kit with modifications. By way of background, innate and recombinant human dimeric fibronectin may have a molecular weight greater than or equal to about 440 kDa to less than or equal to about 500 kDa, and tetrameric streptavidin may have a molecular weight greater than or equal to about 53 kDs to less than or equal to about 55 kDa. In protein-protein coupling chemistry, the mass ratios of the reactants may be adjusted to optimize their molar ratio to maintain their functionality in cell selection and expansion. For example, Bio-Rad LYNX Kits may be designed to couple streptavidin and IgG-class monoclonal antibodies (mAb) at a mass ratio of about 1:1. Herein, the Bio-Rad LYNX Kit reagents may couple fibronectin and streptavidin at a different mass ratio of about 1:3.3 to achieve an effective molar ratio of about 1:3 in a reaction lasting for a period greater than or equal to about 3 hours to less than or equal to about 15 hours at ambient temperature. For example, see Table 1.

TABLE 1
FN:SN Protein Mass Ratio for Mixtures and Coupling

| Protein-Protein | Protein | Protein |
|---|---|---|
| Coupling Stoichiometry | Molar Ratio | Mass Ratio |
| IgG-SN Conjugation | 1:2.7 | 1:1 |
| Bio-Rad LYNX mAb Kit | | (155 kDa:165 kDa) |
| FN-SN Conjugation | 1:3.0 | 1:3.3 |
| Terumo BCT LYNX Modification | | (500 kDa:165 kDa) |

Option 1: Coating with a Mixture of Fibronectin and Streptavidin

This process may involve the reconstitution of lyophilized fibronectin and streptavidin (FN+SN) (e.g., 1:3.3 by mass) in deionized water (DI H₂O) at ambient temperature for about 30 minutes. After the coupling of fibronectin-streptavidin, the mixture volume may be brought up to 100 mL with Longza PBS without Ca²⁺—Mg²⁺ and introduced into the Quantum® System using the “Coat Bioreactor” task for overnight. After bioreactor coating, the excess, unbound protein may be washed out and biotinylated molecule of choice (e.g., cytokine (interleukin or growth factor), epitope, ligand, monoclonal antibody, stains, aptamer, or chemokine) may be introduced into the Quantum® System bioreactor using the “Coat Bioreactor” task for coupling to the FN-SN coating. The resulting fibronectin-streptavidin-bioconjugate protein may be ready for use in cell selection or cell signaling (including differentiation) applications. Other applications may potentially include the coating of preparatory hollow fiber membrane columns or matrixes which may be used for cell selection or differentiation prior to the introduction of cells into the Quantum® System. The exact ratio of fibronectin to streptavidin and coupling methodology may be modified during further development. For example, recombinant or semi-synthetic fibronectin or fibrinogen may be substituted for plasma-derived fibronectin. Extracellular Matrix Proteins such as fibronectin may bind to the polyethersulfones hollow fiber membrane of the Quantum® System bioreactor by virtue of the polarity and hydrogen bonding. SurPASS bound layer studies by Anton Paar show that the Quantum® System polyethersulfones membrane may have a net negative charge under physiological pH 7.2-7.4 conditions. Fibronectin may have a “net positive” charge due to the presence of positively charged amino acid residues such as lysine. In addition, fibronectin may have a naturally adhesive nature due to its glycoprotein structure and specific domains which allow fibronectin to bind to both polyethersulfones and cell membrane integrins.

Option 2: Coating with a Rapid Covalent Coupling of Fibronectin and Streptavidin Using a Modified LYNX Kit

The covalent coupling of fibrinogen to streptavidin, using a similar mass ratio as outlined in “Option 1,” may be another example and may be accomplished using a modified commercially available Bio-Rad LYNX Rapid Streptavidin Conjugation Kit. This kit may use a proprietary linkage modifier and quencher chemistry (LNK161STR, LNK162STR, LNK163STR) to generate a covalent linkage between fibronectin and streptavidin over a period greater than or equal to about 3 hours to less than or equal to about 15 hours. The affinity of the chosen biotinylated molecule to streptavidin, in the covalent coating method, may be similar to the affinity of the biotinylated molecule in the fibrinogen-streptavidin mixture coating method. The advantage of the covalent approach may be the improved stability of the fibrinogen-streptavidin coupling.

### Example 3

Coating a polyethersulfones (PES) hollow fiber membrane bioreactor of a cell expansion system (e.g., Quantum Flex™) may include formulating individual coating agents at specific concentrations and stoichiometric ratios to achieve a layered, hydrogen bonded and functional surface biochemistry.

The polyethersulfones (PES) hollow fiber membrane bioreactor may be primed with sterile-filtered phosphate buffered saline (PBS; 0.0067M PO₄) with calcium (Ca²⁺) or magnesium (Mg²⁺) cations.

Lyophilized human fibronectin (e.g., Corning hFn, 5 mg; Cat. 356008) may be warmed to ambient lab temperature for at least about 30 minutes and reconstituted with about 10 millimeters of sterile deionized (DI) water and let stand or rested for an additional about 30 minutes at ambient temperature. The fibronectin suspension may be further diluted with phosphate buffered saline to a q.s. volume of about 29 milliliters of for introduction into an intracapillary loop of the polyethersulfones (PES) hollow fiber membrane bioreactor, for example, using a default “Coat Bioreactor” Task over a period of greater than or equal to about 4 hours to less than or equal to about 24 hours. Unbound fibronectin may be removed from the polyethersulfones (PES) hollow fiber membrane bioreactor using, for example, a default “IC/EC Washout” Task using 2.5 volumes of phosphate buffered saline.

Lyophilized r-Streptavidin (e.g., ThermoFisher Pierce, 4 mg, Cat. PI21135) may be warmed to ambient temperature for at least about 30 minutes and reconstituted with about 1 milliliters of sterile deionized (DI) water. The streptavidin suspension may be further diluted with phosphate buffered saline to a q.s. volume of about 29 milliliters for introduction into the intracapillary loop of the polyethersulfones (PES) hollow fiber membrane bioreactor using, for example, the default “Coat Bioreactor” Task over a period of greater than or equal to about 4 hours to less than or equal to about 24 hours to achieve a minimum streptavidin addition of about 968 ng/cm² in the polyethersulfones (PES) hollow fiber membrane bioreactor. Unbound Streptavidin may be removed from the polyethersulfones (PES) hollow fiber membrane bioreactor using, for example, a default “IC/EC Washout” Task using 2.5 volumes of phosphate buffered saline.

Lyophilized, biotin-AFDye488 conjugate (e.g., Vector Labs, 1 mg, CCT 1395) may be warmed to ambient temperature and reconstituted with about 1 milliliter of sterile deionized (DI) water. The biotin solution of greater than or equal to about 2 milligrams to less than or equal to about 4 milligrams may be further diluted with phosphate buffered saline to a q.s. volume of about 29 milliliters for introduction into the intracapillary loop of the polyethersulfones (PES) hollow fiber membrane bioreactor using, for example, a default “Coat Bioreactor” Task over a period of greater than or equal to about 4 hours to less than or equal to about 24 hours to achieve 20-fold molar excess. Unbound biotin conjugate may be removed from the polyethersulfones (PES) hollow fiber membrane bioreactor using, for example, a default “IC/EC Washout” Task using 2.5 volumes of phosphate buffered saline. The amount of biotin used may be selected to takes into account the stoichiometric ratio of 4 moles biotin needed to bind to 1 mole of streptavidin plus a 5-molar excess for a total of a 20-molar excess of biotin to streptavidin to assure that sufficient biotin was introduced to occupy the available binding sites on the streptavidin, which was previously coated onto the intracapillary membrane surface.

All of the coating steps may be performed at about 37° C. under active perfusion with the default software tasks and mix-gas conditions of about 5% carbon dioxide (CO₂) and 20% oxygen (O₂) with a balance of nitrogen (N₂). The total phosphate buffered saline volume of the protein/reagent solution may be bioreactor dependent, for example, about 29 millimeters for a Quantum Flex™ cell expansion system small bioreactor or about 100 milliliters for the Quantum Flex™ cell expansion system standard bioreactor. The actual reagents quantities may be adjusted based on the different surface areas of these two polyethersulfones (PES) hollow fiber membrane bioreactors.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for functionalizing a hollow fiber membrane for cell expansion, differentiation, harvesting, activation, or any combination thereof of target cells, the method comprising:
contacting a biotinylated molecule and the hollow fiber membrane, the hollow fiber membrane including an extracellular matrix component, the biotinylated molecule binding to the extracellular matrix component and having an affinity for the target cells.

2. The method of claim 1, wherein the biotinylated molecule is selected from the group consisting of: cytokine, epitope, ligand, monoclonal antibody, stains, aptamer, chemokine, biotin, and combinations thereof.

3. The method of claim 1, wherein the extracellular matrix component is selected from the group consisting of: fibronectin, vitronectin, fibrinogen, collagen, laminin, streptavidin, truncations thereof, and combinations thereof.

4. The method of claim 1, wherein the extracellular matrix component includes an extracellular protein-streptavidin coupling, an extracellular protein of the extracellular protein-streptavidin coupling binding to a target surface of the hollow fiber membrane, and streptavidin of the extracellular protein-streptavidin coupling binding to the biotinylated molecule.

5. The method of claim 4, wherein the hollow fiber membrane has extracellular protein-streptavidin coupling has a mass ratio of the streptavidin to the biotinylated molecule of greater than or equal to about 1:3 to less than or equal to about 1:9.

6. The method of claim 4, wherein the extracellular protein-streptavidin coupling includes a fibronectin-streptavidin coupling, the fibronectin having a molecular weight greater than or equal to about 440 kDa to less than or equal to about 500 kDa, and the streptavidin having a molecular weight greater than or equal to about 53 kDa to less than or equal to about 55 kDa.

7. The method of claim 6, wherein the method further includes preparing the fibronectin-streptavidin coupling.

8. The method of claim 7, wherein the preparing of the fibronectin-streptavidin coupling includes reconstituting lyophilized fibronectin with streptavidin by immerging the lyophilized fibronectin and streptavidin in water.

9. The method of claim 7, wherein the preparing of the fibronectin-streptavidin includes covalently coupling the fibronectin and the streptavidin.

10. The method of claim 4, wherein the method further includes preparing the extracellular protein-streptavidin coupling, the preparing of the extracellular protein-streptavidin coupling including
contacting the extracellular protein and a precursor hollow-fiber membrane, and
after the contacting of the extracellular protein and the precursor hollow-fiber membrane, contacting the streptavidin and the precursor hollow-fiber membrane including the extracellular protein to form the extracellular protein-streptavidin coupling and the hollow fiber membrane including the extracellular matrix component, the extracellular protein-streptavidin coupling including both classical and non-classical hydrogen bonding between the extracellular protein and the streptavidin.

11. The method of claim 10, wherein the extracellular protein is contacted with the precursor hollow fiber membrane for a first period greater than or equal to about 4 hours to less than or equal to about 24 hours.

12. The method of claim 11, wherein the method further includes, after the first period and before the contacting of the streptavidin and the precursor hollow-fiber membrane, washing the precursor hollow fiber membrane to remove any unreacted and excess portions of the extracellular protein.

13. The method of claim 10, wherein the streptavidin is contacted to the precursor hollow fiber membrane for a second period greater than or equal to about 4 hours to less than or equal to about 24 hours.

14. The method of claim 13, wherein the method further includes, after the second period and before the contacting of the biotinylated molecule and the hollow fiber membrane including the extracellular matrix component, washing the hollow fiber membrane including the extracellular matrix component to remove any unreacted and excess portions of the streptavidin.

15. The method of claim 1, wherein the method further includes contacting the extracellular matrix component and a precursor hollow fiber membrane to form the hollow fiber membrane including the extracellular matrix component.

16. The method of claim 15, wherein the extracellular matrix component is contacted with a surface of the hollow fiber membrane for a period greater than or equal to about 4 hours to less than or equal to about 24 hours prior to the contacting of the biotinylated molecule and the surface.

17. The method of claim 16, wherein after the period and prior to the contacting of the biotinylated molecule and the surface, the method further includes washing the hollow fiber membrane to remove any unreacted and excess portions of the extracellular matrix component.

18. A method for functionalizing a hollow fiber membrane for cell expansion, differentiation, harvesting, activation, or any combination thereof of target cells, the method comprising:
contacting an extracellular protein and a surface of a hollow fiber membrane;
after the contacting of the extracellular protein and the surface of the hollow fiber membrane, contacting streptavidin and the surface of the hollow fiber membrane, the streptavidin forming extracellular protein-streptavidin coupling with the extracellular protein, the extracellular protein-streptavidin coupling including both classical and non-classical hydrogen bonding between the extracellular protein and the streptavidin; and
after the contacting of the streptavidin and the surface of the hollow fiber membrane, contacting a biotinylated molecule and the surface of hollow fiber membrane, the biotinylated molecule binding to the extracellular protein of the extracellular protein-streptavidin coupling and having an affinity for the target cells.

19. The method of claim 18, further including, after the contacting of the extracellular protein and the surface of the hollow fiber membrane and before the contacting of the streptavidin and the surface of the hollow fiber membrane, washing the surface of hollow fiber membrane to remove any unreacted and excess portions of the extracellular protein.

20. The method of claim 18, further including, after the contacting of the streptavidin and the surface of the hollow fiber membrane and before the contacting of the biotinylated molecule and the surface of the hollow fiber membrane, washing the surface of hollow fiber membrane to remove any unreacted and excess portions of the streptavidin.
